(19) United States
(12) Patent Application Publication
(10) Pub. No.: US 2019/0367868 A1
(43) Pub. Date: Dec. 5, 2019

(54) NEURONS AND COMPOSITIONS AND METHODS FOR PRODUCING THE SAME

(71) Applicant: President and Fellows of Harvard College, Cambridge, MA (US)

(72) Inventors: Lee L. Rubin, Wellesley, MA (US); Alessandra Rigamonti, Stockholm (SE)

(21) Appl. No.: 16/067,778

(22) PCT Filed: Dec. 30, 2016
(86) PCT No.: PCT/US2016/069585

§ 371 Date: Jul. 2, 2018

Related U.S. Application Data
(60) Provisional application No. 62/273,819, filed on Dec. 31, 2015.
(60) Provisional application No. 62/342,566, filed on May 27, 2016.

(51) Int. Cl. C12N 5/00; C12N 5/0793; G01N 33/50

(52) U.S. Cl. CPC C12N 5/0018; C12N 5/0619; G01N 33/5091; C12N 5/0062; C12N 2527/00; C12N 2500/30; C12N 2501/415; C12N 2501/13; C12N 2506/02; C12N 2506/45; C12N 2513/00; C12N 2501/999; G01N 2800/2814

(57) ABSTRACT

Disclosed herein are methods for generating functional motor neurons, compositions including functional motor neurons, and methods for screening for neurodegenerative diseases.

### RELATED APPLICATIONS

This application is a national stage filing under 35 U.S.C. 371 of International Application No. PCT/US2016/069585, filed Dec. 30, 2016, which claims the benefit of U.S. Provisional Application No. 62/273,819 filed Dec. 31, 2015, and U.S. Provisional Application No. 62/342,566 filed May 27, 2016. The entire teachings of the above applications are incorporated herein by reference. International Application No. PCT/US2016/069546 was published under PCT Article 21(2) in English.

### BACKGROUND OF THE INVENTION

With the seminal discovery of human pluripotent stem cells (hPSCs) (Thomson et al., 1998; Takahashi et al., 2007), human cells that would be difficult or impossible to obtain can be produced using in vitro cell culture techniques. This in turn has raised hopes that hPSCs can be used to study and treat different forms of disease, including neurological and neuropsychiatric disorders (Dolmetsch and Geschwind, 2011; Fox et al., 2014; Han et al., 2011; Imaizumi and Okano, 2014; Kanning et al., 2010; Liu and Zhang, 2010). However, a key step in the utilization of hPSCs for these purposes is the ability to obtain cell types of interest. This has often proven to be challenging for several reasons, among them are neural diversity, culture to culture and line to line variability, and limitations on large-scale cell production.

Several methods have been described to obtain neurons of specific subtypes through differentiation of hPSCs, either via formation of three-dimensional (3D) embryoid bodies (EB) or using monolayers as starting materials (Amoroso et al., 2013; Boissart et al., 2013; Boulting et al., 2011; Eiraku and Sasai, 2012; Eiraku et al., 2008; Espuny-Camacho et al., 2013; Hu and Zhang, 2009; Kim et al., 2014; Li et al., 2009; Qu et al., 2014; Shi et al., 2012; Zeng et al., 2010). Alternative approaches include transcriptional programming, where the forced overexpression of a cocktail of transcription factors instruct PSCs, fibroblasts, or other cell populations to adopt a specific neuronal fate (Hester et al., 2011; Vierbuchen et al., 2010). These methods have provided important insights into human neurogenesis and the pathogenesis of neurodevelopmental disorders, but they have important limitations. For instance, EB-based protocols generally have comparatively low efficiencies (10%-40%) and require a relatively long time in culture to generate functional motor neurons. In addition, the neurons generated often require cellular feeder layers to survive for longer times in culture (Hu and Zhang, 2009, Boulting et al. 2011, Amoroso et al. 2013). Moreover, the EB method results in the formation of spheres of cells varying in size and shape, leading to differences in the kinetics and efficiency of differentiation within individual plates and from experiment to experiment. Monolayer-based protocols for generation of both cortical and motor neurons have been published, with recent work describing improved efficiencies (Qu et al., 2014). However, a disadvantage of this adherent monolayer-based protocol is that the neurons need to be passaged and successful long term culture after replating has not been described.

Another common theme in the field has been the problem of obtaining functional cells from hPSCs. It has been shown that maintaining differentiated cells in culture can be challenging, thereby precluding experiments studying aspects of cellular functions that take longer times to manifest (Bellin et al. 2012, Grskovic et al. 2011).

Human pluripotent stem cells (hPSCs) offer a renewable source of cells that can be expanded indefinitely and differentiated into virtually any type of cell in the human body, including neurons. This opens up unprecedented possibilities to study cell and developmental biology and cellular pathology of the nervous system, provides a platform for the screening of chemical libraries that affect these processes, and offers a potential source of transplantable cells for regenerative approaches to neurological disease. However, defining protocols that permit a large number and high yield of neurons has proven difficult.

### SUMMARY OF THE INVENTION

There is a need for differentiation protocols for the generation of distinct subtypes of neurons in a highly reproducible manner, with minimal experiment-to-experiment variation. The present invention is directed towards methods for production (e.g., large scale production) of cortical neurons and motor neurons from multiple cell lines (e.g., human embryonic stem cells (hESCs) or human induced pluripotent stem cells (hiPSCs)). These neurons form synapses with neighboring cells, exhibit spontaneous electrical activity, and respond appropriately to depolarization. For example, hPSC-derived neurons exhibit a high degree of maturation and survive in culture up to 4-5 months, even without glial cell feeder layers.

In some embodiments, the present invention is directed to methods of generating functional neurons from stem cells, the methods comprising contacting a population of stem cells in spin culture with at least one differentiation medium to induce the differentiation of at least one sphere in the spin culture into a functional neuron differentiation sphere.

In some aspects, the population of stem cells are spin cultured at a rotation rate of about 25-150 rpm. In some aspects, the population of stem cells are spin cultured at a rotation rate of about 50-100 rpm. In some aspects, the population of stem cells are spin cultured at a rotation rate of about 60-80 rpm. In some aspects, the population of stem cells are spin cultured at a rotation rate of about 70 rpm. In some aspects, the population of stem cells are spin cultured at a rotation rate of about 5 rpm, 10 rpm, 15 rpm, 20 rpm, 25 rpm, 30 rpm, 35 rpm, 40 rpm, 45 rpm, 50 rpm, 55 rpm, 60 rpm, 65 rpm, 70 rpm, 75 rpm, 80 rpm, 85 rpm, 90 rpm, 95 rpm, 100 rpm, 105 rpm, 110 rpm, 115 rpm, 120 rpm, 125 rpm, 130 rpm, 135 rpm, 140 rpm, 145 rpm, or 150 rpm.

In some embodiments, the functional neuron differentiation sphere is a motor neuron differentiation sphere. In some embodiments, the functional neuron differentiation sphere is a cortical neuron differentiation sphere. In some embodiments, the differentiation medium is a neural induction medium. In some embodiments, the differentiation medium includes a supplemental agent. In some embodiments, the supplemental agent is selected from the group consisting of retinoic acid, brain-derived neurotrophic factor, smoothened agonist, DAPT and combinations thereof.

In some embodiments, the present invention is directed to methods of generating functional cortical neurons from stem cells, the methods comprising contacting a population of stem cells in spin culture with at least one differentiation medium to induce the differentiation of at least one sphere in the spin culture into a functional cortical neuron differentiation sphere.

In some embodiments, the at least one differentiation medium includes dual SMAD inhibition. In some embodiments, a first differentiation medium includes a KSR medium. In certain embodiments, the first differentiation medium includes a supplemental agent. In certain embodiments, the supplemental agent is a Wnt signaling inhibitor. In some embodiments, a second differentiation medium includes a neural induction medium. In some embodiments, the population of stem cells in spin culture are contacted with a KSR medium including a Wnt signaling hibitor. In certain embodiments, the population of stem cells in spin culture are further contacted with a neural induction medium. In some embodiments, the methods further include maintaining the functional cortical neuron spheres in a neurobasal medium.

In some embodiments, the present inventions are directed to methods of generating functional motor neurons from stem cells, the methods comprising contacting a population of stem cells in spin culture with at least one differentiation medium to induce the differentiation of at least one sphere in the spin culture into a functional motor neuron differentiation sphere.

In some embodiments, the at least one differentiation medium includes dual SMAD inhibition. In some embodiments, a first differentiation medium includes a KSR medium. In certain embodiments, the first differentiation medium includes a supplemental agent. In certain embodiments, the supplemental agent is selected from the group consisting of retinoic acid, brain-derived neurotrophic factor, smoothened agonist, DAPT and combinations thereof. In some embodiments, a second differentiation medium includes a neural induction medium. In some embodiments, the population of stem cells in spin culture are contacted with a KSR medium including at least one supplemental agent selected from the group consisting of retinoic acid, brain-derived neurotrophic factor, smoothened agonist, and combinations thereof. In certain embodiments, the population of stem cells in spin culture are further contacted with a neural induction medium including at least one supplemental agent selected from the group consisting of retinoic acid, brain-derived neurotrophic factor, smoothened agonist, DAPT and combinations thereof. In some embodiments, the methods further include maintaining the functional motor neuron spheres in a neurobasal medium including at least one supplemental agent selected from the group consisting of brain-derived neurotrophic factor, glial cell-derived neurotrophic factor, ciliary neurotrophic factor and combinations thereof.

In some embodiments, the present inventions are directed to methods for screening for neurodegenerative diseases, the methods comprising generating a neuron composition comprising a functional neuron from patient-derived induced pluripotent stem cells, and screening for dysregulation of spontaneous activity or defects of stimulus-induced activity.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F demonstrate some three-dimensional neuronal spheres from hPSC lines adopt a predominantly cortical identity by default. FIG. 1A depicts a schematic illustration of one differentiation strategy for obtaining cortical neurons from three-dimensional spheres. FIG. 1B shows a representative bright field image of HUES8-derived cortical spheres at day 15 of differentiation. FIG. 1C shows representative images of immunohistochemistry on sections of BJSiPS-derived cortical spheres at day 50 of differentiation, stained with antibodies specific for MAP2 (blue), TBR1 (red), and CTIP2 (green). Scale bar represents 200 μm. FIGS. 1D-1F provide representative results from a BJSiPS-derived cortical sphere cleared by SeeDB at day 50 of differentiation stained with antibodies specific for TBR1 (red), CTIP2 (green), and SATB2 (blue), and imaged by light-sheet fluorescence microscopy. Photos in FIG. 1D show 3D images of the same sphere from two different angles. Schematic images in FIG. 1E illustrate the virtual section of the sphere. The low magnification 3D image in FIG. 1E (left panel) shows the distribution of the neurons expressing markers of different cortical subtypes inside the sphere. FIG. 1E (right panel) is a higher magnification image of the boxed area in FIG. 1E (left panel). Schematic illustrations in FIG. 1F indicate how the sphere was virtually processed to generate a partially sectioned sphere.

FIGS. 2A-2E demonstrate some neurons from dissociated cortical spheres maintain the differentiated phenotype in culture and express synaptic proteins. FIGS. 2A-2B show representative images of immunocytochemistry of neurons from BJSiPS-derived cortical spheres dissociated at day 40, plated and cultured for 7 days (FIG. 2A) and 22 days (FIG. 2B) after dissociation. Cells were stained using antibodies specific for MAP2 (purple), CTIP2 (green), and TBR1 (red) in FIG. 2A, and antibodies specific for MAP2 (purple) and SATB2 (green) in FIG. 2B. Nuclei were counterstained with DAPI. Scale bars represent 50 μm. FIGS. 2C-2E provide quantification of relative proportions of cortical subtypes obtained from experiments in different hPSC lines. Cells were dissociated at day 40, and plated and processed for immunocytochemistry with the indicated antibodies 7 and 22 days after dissociation. Error bars indicate standard deviation from three independent experiments.

FIGS. 3A-3F demonstrate dissociated cortical neurons are spontaneously active. FIG. 3A provides representative immunocytochemistry of dissociated neurons from HUES8-derived cortical spheres stained with antibodies specific for MAP2 (blue), SYNAPSIN1 (green), and GLUR1 (red). Images in FIG. 3A (right panel) constitute a higher magnification of the boxed area in FIG. 3A (left panel). FIG. 3B provides representative results from experiments where BJSiPS-derived cortical spheres were dissociated at day 40. Three days post dissociation, the plated neurons were transduced with AAV:GCaMP6, and time-lapse microscopy was employed to record fluctuations in fluorescence over time (20, 40, 60, and 75 days after dissociation). FIGS. 3C-3F provide electrophysiological recordings of HUES8-derived cortical neurons 6 weeks after dissociation from spheres. FIG. 3C provides representative traces showing voltage-gated sodium and potassium currents. FIG. 3D shows cortical neurons fired action potential upon current injection (30 pA). FIG. 3E provides representative traces showing spontaneous firing of action potentials. FIG. 3F provides samples traces showing spontaneous excitatory postsynaptic currents (EPSCs, fast decay) and inhibitory postsynaptic currents (IPSCs, slow decay) with a holding potential of −70 mV.

FIGS. 4A-4C demonstrate dissociated cortical neurons respond to depolarization. FIG. 4A shows representative images of immunocytochemistry of HUES8-derived cortical neurons 20 days after dissociation from spheres before and after stimulation by KCl. Cells were stained using antibodies specific for MAP2 (purple), CTIP2 (red) and phosphorylated forms of CREB (green). Nuclei were counterstained with DAPI. Scale bars represent 50 μm. FIG. 4B provides quantification of a relative proportion of neurons staining positive in immunocytochemistry experiments using an antibody specific for phospho-CREB. Error bars indicate standard deviation from three independent experiments. FIG. 4C provides quantification of expression of NPAS4, cFOS, ARC, and BDNF following stimulation by KCl by qRT-PCR. Error bars indicate standard deviation from three independent experiments.

FIGS. 5A-5F demonstrate some three-dimensional neuronal spheres from human PSC lines differentiate into motor neurons in the presence of patterning factors. FIG. 5A provides a schematic illustration of a differentiation strategy to obtain motor neurons from three-dimensional spheres. FIG. 5B provides representative images of immunohistochemistry on sections of 1016A-derived spheres differentiated towards the motor neuron fate and stained with antibodies specific for MAP2 (red) and ISL1 (green) at day 20 of differentiation. Nuclei were counterstained with DAPI (blue). Scale bar represents 50 μm. FIG. 5C provides representative images of immunocytochemistry of 1016A-derived motor neurons from dissociated motor neuron spheres plated and cultured for 7 days after dissociation. Cells were stained using antibodies specific for MAP2 (purple) and ISL1 (green). Nuclei were counterstained with DAPI (blue). Scale bar represents 50 μm. FIG. 5D depicts quantification of the relative proportion of the different motor neurons subtypes obtained from experiments in different hPSC lines. Cells were dissociated at day 15, plated, cultures for 1 week and processed for immunocytochemistry with an antibody specific for ISL1. FIG. 5E provides representative immunocytochemistry of 1016A-derived neurons from spheres stained with antibodies specific for MAP2 (purple), ISL1 (green), and ChAT (red). Nuclei were counterstained with DAPI. Scale bar represents 50 μm. FIG. 5F depicts quantification of the relative proportion of ChAT positive cells obtained from experiments in two different hPSC lines. Cells were dissociated at day 15, plated, cultured for 1 week and processed for immunocytochemistry with an antibody specific for ChAT. Error bars indicate standard deviation from three independent experiments.

FIGS. 6A-6E demonstrate some dissociated motor neurons are spontaneously active. FIG. 6A shows representative immunocytochemistry of 1016A-derived neurons from dissociated motor neuron spheres dissociated at day 15, cultured for 1 week and stained with antibodies specific for MAP2 (green) and SYNAPSIN1 (red). Nuclei were counterstained with DAPI (blue). FIG. 6A (right panel) is an image captured at higher magnification of the boxed area in FIG. 6A (left panel). Scale bar represents 20 μm. FIG. 6B shows representative immunocytochemistry of HUES8-motor neurons spheres seeded at day 15 on top of cultures of human myotubes. The co-culture is stained after 1 week with antibodies for SMI32 (green) and fluorescently labeled alpha-bungarotoxin (BTX) (red). FIG. 6B′ is an image captured at higher magnification of the boxed area in B. FIG. 6C shows representative results from experiments where HUES8-derived motor neurons were dissociated from spheres at day 15 and transduced with AAV: GCaMP6 after 3 days. Time-lapse microscopy was employed to record fluctuations in fluorescence over time 1 and 3 weeks after transduction. FIG. 6D depicts electrophysiological recordings of HUES8-derived neurons from motor spheres analyzed 12 weeks after dissociation. The left panel identifies representative traces showing spontaneous action potential firing. The right panel identifies representative traces showing evoked action potential firing upon current injection from 0 to 90 pA in 10 pA increments. FIG. 6E provides representative traces showing normalized fluorescence intensity changes of GCaMP6s after GABA and AMPA (both at 100 μM) application in HUES9-derived motor neurons cultured for 3 weeks after sphere dissociation.

FIGS. 7A-7G demonstrate some sphere-derived human motor neurons survive longer in culture and maintain functionality throughout extended time in culture. FIG. 7A shows representative bright field images of HUES9 EBs (left) and HUES9 motor neuron spheres generated by spin culture (right) at day 6 of differentiation. FIG. 7B shows representative images of immunocytochemistry of HUES9-derived motor neurons obtained through EB formation (top panel) or spin culture differentiation (bottom panel) stained after 7 days of culture with antibodies against MAP2 (purple), ISL1 (green) and ChAT (red). Nuclei were counterstained with DAPI (blue). FIGS. 7C-7E provide an analysis of expression levels of ISL1, HB9 and ChAT by qRT-PCR in cells undergoing differentiation through EB formation (green bars) or spin culture (red bars) at different time points. Error bars indicate standard deviation from three independent experiments.

FIG. 7F provides representative images of dissociated H9ISL1RFP-reporter line motor neurons from the EBs (top panel) and spin spheres (bottom panel), plated under feeder-free conditions and imaged after 10, 27, 35 and 42 days of culture. Scale bar represents 50 μm. FIG. 7G shows representative results from experiments where H9ISL1RFP-reporter line motor neurons derived using EBs (top panel) or spin sphere (bottom panel) methodology were dissociated and transduced with AAV: GCaMP6 3 days after dissociation. Time-lapse microscopy was employed to record fluctuations in fluorescence over time (10, 27, 35 and 42 days after transduction).

FIGS. 8A-8C demonstrate prior to differentiation, the size and general appearance of some hPSC spheres are homogenous, and some cells express the pluripotency markers OCT4 and NANOG. At day 50 of differentiation, images obtained by light-sheet fluorescence microscopy show the same cortical layer organization in different BJSiPS-derived cortical spheres. FIG. 8A shows bright field images of three-dimensional HUES8 hPSC spheres growing in spin culture. Scale bars represent 200 μm (left) and 100 μm (right). FIG. 8B shows representative images of immunohistochemistry of sections of HUES8 hPSC spheres grown in spin culture stained with antibodies specific for the pluripotency marker OCT4 (green) and NANOG (green). Nuclei were counterstained with DAPI (blue). Scale bar represents 200 μm. FIG. 8C shows representative results from two different cortical spheres cleared by SeeDB, stained with antibodies specific for TBR1 (red), CTIP2 (green), and SATB2 (blue), and imaged by light-sheet fluorescence microscopy.

FIG. 9 demonstrates during differentiation some spin culture spheres showed a rapid loss of expression of pluripotency genes, and gradual establishment of persistent expression of cortical progenitor and neuronal markers. Quantification of expression of the pluripotency marker OCT4, neural progenitor marker OTX1, cortical progenitor marker FOXG1, and the pan-neuronal and cortical markers MAP2, TBR1, CTIP2, CUX1 and SATB2 by qRT-PCR analysis of RNA collected from harvested spheres at different time points. Error bars indicate standard deviation from three independent experiments.

FIGS. 10A-10D demonstrate immunohistochemistry shows the presence of the neural progenitor markers OTX1 and 2 of the proliferative marker Ki67 in the culture. FIGS. 10A and 10B show representative images of immunocytochemistry of neurons from BJSiPS-derived cortical spheres dissociated at day 40, plated and cultured for 7 (top panel) and 22 days (bottom panel) after dissociation. Cells were stained using antibodies specific for MAP2 (green), Ki67 (red) and OTX1-2 (yellow). Nuclei were counterstained with DAPI. Scale bars represent 50 μm. FIGS. 10C and 10D provide quantification of the relative proportion of cells expressing the neural progenitor markers OTX1 and 2 and the proliferative marker Ki67 obtained from experiments in different hPSC lines. Cells were dissociated at day 40, plated and processed for immunocytochemistry with the indicated antibodies 7 and 22 days after dissociation. Error bars indicate standard deviation from two independent experiments.

FIGS. 11A-11C demonstrate some three-dimensional neuronal spheres from the HUES8 hPSC lines adopt a predominantly cortical identity in absence of patterning factors, exhibit immunoreactivity for synaptic proteins and respond to depolarization. FIG. 11A provides representative images of immunohistochemistry of sections of cortical spheres stained at day 50 of differentiation with antibodies specific for TBR1 (red), CTIP2 (green) and SATB2 (blue). Scale bar represents 200 μm. Nuclei were counterstained with DAPI (blue). FIG. 11B provides representative images of immunohistochemistry of sections of cortical spheres stained at day 50 of differentiation with antibodies specific for MAP2 (green) and SYNAPSIN1 (red). FIG. 11C provides quantification of expression of the activity-induced genes NPAS4, cFOS, ARC, and BDNF following stimulation of spheres at day 60 of differentiation by KCl. Levels of transcribed mRNA from the indicated genes were quantified by qRT-PCR. Error bars indicate standard deviation from two independent experiments.

FIG. 12 demonstrates by supplying RA and SHH in exact concentrations, some spin culture spheres differentiated towards the motor neuron fate. Quantification of expression of the pluripotency marker OCT4, neuronal progenitor SOX1, the pan-neuronal and cortical markers MAP2, ISL1, HB9 and the biosynthetic enzyme ChAT by qRT-PCR analysis of RNA collected at different time points. Error bars indicate standard deviation from two independent experiments.

FIGS. 13A-13D demonstrate some three-dimensional motor spheres express the appropriate biosynthetic enzyme ChAT and form synapses. FIGS. 14A and 14B show representative images of immunohistochemistry of sections of 1016A-derived spheres differentiated towards the motor neuron fate and stained at day 20 of differentiation with antibodies specific for ISL1 (green) and ChAT (red), and nuclei were counterstained with DAPI (blue) (FIG. 13A), and for MAP2 (blue), SYNAPSIN1 (red) and ISL2 (green) (FIG. 13B). Scale bar represents 100 μM. FIG. 13D shows representative images of immunocytochemistry of dissociated neurons from motor neuron spheres before and after stimulation by KCl. Cells were stained using antibodies specific for MAP2 (purple) and the phosphorylated forms of CREB (green). Motor neurons were identified using a previously described genetically modified hESC line permitting lineage-tracing of ISL1-expressing cells through Cre-dependent expression of RFP. Scale bars represent 100 μm. 50 μm. FIG. 13D provides quantification of the relative proportion of motor neurons stained positive for phospho-CREB. Error bars indicate standard deviation from two independent experiments.

FIGS. 14A-14K demonstrate some spin culture motor spheres are more homogeneous in size and spin derived-motor neurons exhibit some features of a more mature phenotype compared to EB-derived motor neurons. FIG. 14A provides representative traces showing voltage-gated sodium and potassium currents recorded from HUES9-derived motor neurons derived from EBs and spin spheres 10-12 days after dissociation. The membrane potential was depolarized from −60 mV to 50 mV in 10 mV increments with a holding potential of −70 mV. FIG. 14B shows current-voltage plots of sodium currents recorded from HUES9-derived motor neurons at 10-12 and 22-23 days after dissociation. FIG. 14C provides bar graphs showing the peak amplitude of sodium currents (mean±SEM). *p<0.05, **p<0.01 (Student's t-test). FIG. 14D provides current-voltage plots of potassium currents recorded from HUES9-derived motor neurons. FIG. 14E provides bar graphs showing the peak amplitude of potassium currents (mean±SEM). **p<0.01 (Student's t-test). FIG. 14F depicts representative traces showing evoked action potentials in response to 90 pA current injection recorded from HUES9-derived human motor neurons 10-12 days after dissociation. The membrane potential was held at −60 mV. Bar graphs showing the half-width of action potentials (mean±SEM, the width at half-maximal peak amplitude) (left) and the height of action potentials (right) (mean±SEM, peak relative to after hyperpolarization).*p<0.05, **p<0.01 (Student's t-test). FIG. 14G provides bar graphs showing the peak amplitude of GABA-induced GCaMP6 fluorescence increases (mean±SEM). *p<0.05, ***p<0.001 (Student's t-test). FIG. 14H provides bar graphs showing the peak amplitude of AMPA-induced GCaMP6 fluorescence increases (mean±SEM). FIG. 14I shows bright field images of three-dimensional HUES9-derived motor neurons spheres grown under spin culture conditions (top panel) and cultured as EBs in low attachment tissue culture plates (bottom panel) at different time points. Scale bars represent 100, 500 and 250 μm (top; left to right) and 500, 250, 250 μm (bottom; left to right). FIG. 14J provides quantification of the relative size of motor neurons spheres and EBs obtained from experiments in different hPSC lines at different time points. Error bars indicate standard deviation of the size of the spheres and EBs at each time point. FIG. 14K depicts coefficient of variation (CV) documenting the variance in size for spin spheres and EBs at different time points and from two different hPSC lines.

### DETAILED DESCRIPTION OF THE INVENTION

The work described herein shows that the described method, based on differentiation of three-dimensional hPSC spheres maintained in suspension in spinner flasks (hereafter referred to as spin culture), can produce a high purity and large absolute number of cells, and has the potential to make functional neurons that can be maintained in culture for extended periods of time. In some embodiments, this method has been applied towards the production of both cortical neurons of multiple types and towards spinal cord motor neurons. For both neuronal subtypes, not only was the expression of appropriate marker genes documented, but several characteristics of mature neurons were also documented. The obtained neurons exhibited certain characteristics, including, but not limited to, responding robustly to depolarization, form synapses as determined by punctate staining with antibodies against established synaptic proteins, and exhibit spontaneous neural activity assessed by electrophysiology and by fluctuations in intracellular Ca2+ levels.

In some aspects, neurons generated from spheres exhibit increased survival after dissociation compared to EB-derived neurons, and, in some aspects, can be obtained in culture without glial feeder layers for up to 5 months without compromising their functional properties. In some aspects, the described protocol generates large numbers of functional neurons that can reach a high degree of maturation, and offer an improved cellular source of human neurons for disease modeling and drug screening.

### Definitions

For convenience, certain terms employed herein, in the specification, examples and appended claims are collected here. Unless otherwise defined, all technical and scientific terms used herein have the same meaning as commonly understood by one of ordinary skill in the art to which this invention belongs.

The term “differentiated cell” is meant to include any primary cell that is not, in its native form, pluripotent as that term is defined herein. Stated another way, the term “differentiated cell” refers to a cell of a more specialized cell type derived from a cell of a less specialized cell type (e.g., a stem cell such as an induced pluripotent stem cell) in a cellular differentiation process.

As used herein, the term “somatic cell” refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell type: internal organs, skin, bones, blood, and connective tissue are all made up of somatic cells. In some embodiments the somatic cell is a “non-embryonic somatic cell,” by which is meant a somatic cell that is not present in or obtained from an embryo and does not result from proliferation of such a cell in vitro. In some embodiments the somatic cell is an “adult somatic cell,” by which is meant a cell that is present in or obtained from an organism other than an embryo or a fetus or results from proliferation of such a cell in vitro.

As used herein, the term “adult cell” refers to a cell found throughout the body after embryonic development.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “organoid” refers to a three-dimensional organ-bud grown in vitro and in isolation from an intact organism. Organoids may be derived from stem cells (e.g., embryonic stem cells, induced pluripotent stem cells, etc.). Various organoids may be formed including, but not limited to, cerebral organoids, thyroid organoids, intestinal organoids, testicular organoids, hepatic organoids, pancreatic organoids, gastric organoids, epithelial organoids, lung organoids, kidney organoids, retina organoids, inner ear organoids, and pituitary organoids.

The terms “cerebral organoid”, “3D brain tissue” or “brain organoid” are used interchangeably herein and refer to organoids that have anatomical features that resemble mammalian brains. The cerebral organoids may include synthesized tissues that contain several types of nerve cells. Cerebral organoids can be produced using human pluripotent stem cells (hPSCs). The general methodology for producing cerebral organoids includes culturing hPSCs under conditions suitable for the development of an embryoid body. The cell culture is then induced to form a neuroectoderm and the neuroectoderm is grown in a protein matrix. The neuroectoderm begins to proliferate and grow and is transferred to a tissue culture vessel where the cerebral organoids will continue to develop. Cerebral organoids may differentiate into one or more of various neural tissue types, such as the optic cup, hippocampus, ventral parts of the telencephalon and dorsal cortex.

The term “phenotype” refers to one or a number of total biological characteristics that define the cell or organism under a particular set of environmental conditions and factors, regardless of the actual genotype.

The term “pluripotent” as used herein refers to a cell with the capacity to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and refers to a pluripotent stem cell artificially derived (e.g., induced or by complete reversal) from a non-pluripotent cell, typically an adult somatic cell, for example, by inducing a forced expression of one or more genes.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art. As used herein, the term “pluripotent stem cell” includes embryonic stem cells, induced pluripotent stem cells, placental stem cells, etc.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as an ectodermal stem cell), which in turn can differentiate into other types of precursor cells further down the pathway (such as a neural ectodermal cell), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting at least one embryoid body or a precursor thereof with a differentiation medium or agent) is intended to include incubating the differentiation medium and/or agent and the cell together in vitro (e.g., adding the differentiation medium or agent to cells in culture). In some embodiments, the term “contacting” is not intended to include the in vivo exposure of cells to the compounds as disclosed herein that may occur naturally in a subject (i.e., exposure that may occur as a result of a natural physiological process). The step of contacting at least one embryoid body or a precursor thereof with a differentiation medium or agent as in the embodiments related to the production of neural tissue can be conducted in any suitable manner. For example, the cells may be treated in adherent culture, or in suspension culture. In some embodiments, the cells are treated in conditions that promote cell clustering. The disclosure contemplates any conditions which promote cell clustering. Examples of conditions that promote cell clustering include, without limitation, suspension culture in low attachment tissue culture plates, spinner flasks, aggrewell plates. In some embodiments, the inventors have observed that clusters have remained stable in media containing 10% serum. In some embodiments, the conditions that promote clustering include a low serum medium.

It is understood that the cells contacted with a differentiation medium and/or agent can also be simultaneously or subsequently contacted with another agent, such as a growth factor or other differentiation agent or environments to stabilize the cells, or to differentiate the cells further.

Similarly, at least one insulin-positive endocrine cell or a precursor thereof can be contacted with at least one differentiation medium or agent and then contacted with at least another differentiation medium or agent. In some embodiments, the cell is contacted with at least one differentiation medium or agent, and the contact is temporally separated, and in some embodiments, a cell is contacted with at least one differentiation medium substantially simultaneously. In some embodiments, the cell is contacted with at least two, at least three, at least four, at least five, at least six, at least seven, at least eight, at least nine, or at least 10 differentiation mediums or agents.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other. In some embodiments, a cell line comprises a neural cell or neural tissue described herein.

The terms “feeder cells” or “feeders” refer to cells of one type that are co-cultured with cells of another type, to provide an environment in which the cells of the second type can grow. The feeder cells are optionally from a different species as the cells they are supporting. In some aspects, a culture or cell population may be referred to as “feeder free”, meaning the composition is essentially free of feeder cells.

The term “growth environment” refers to an environment in which cells of interest will proliferate or differentiate in vitro. Features of the environment include the medium in which the cells are cultured, the temperature, the partial pressure of O2 and CO2, and a supporting structure (such as a substrate on a solid surface) if present.

The term “nutrient medium” refers to a medium for culturing cells containing nutrients that promote proliferation. The nutrient medium may contain any of the following in an appropriate combination: isotonic saline, buffer, amino acids, antibiotics, serum or serum replacement, and exogenously added factors. A “conditioned medium” is prepared by culturing a first population of cells in a medium, and then harvesting the medium. The conditioned medium (along with anything secreted into the medium by the cells) may then be used to support the growth of a second population of cells.

The term “embryoid bodies” is synonymous with “aggregate bodies”. The terms refer to aggregates of differentiated and undifferentiated cells that appear when pPS cells overgrow in plated or suspension cultures.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The terms “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. It should be appreciated that the term genetically modified is intended to include the introduction of a modified RNA directly into a cell (e.g., a synthetic, modified RNA). Such synthetic modified RNAs include modifications to prevent rapid degradation by endo- and exo-nucleases and to avoid or reduce the cell's innate immune or interferon response to the RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation, the modification is not suitable for the methods and compositions described herein.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL world-wide web address of: “ncbi.nlm nih.gov” for these programs. In a specific embodiment, percent identity is calculated using BLAST2 with default parameters as provided by the NCBI.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” as used herein describes a cell with a common ancestry or cells with a common developmental fate. For example, in the context of a cell that is of ectoderm origin or is “ectodermal linage” this means the cell was derived from an ectoderm cell and can differentiate along the ectoderm lineage restricted pathways.

As used herein, the term “xenogeneic” refers to cells that are derived from different species.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “functional fragments” as used herein is a polypeptide having amino acid sequence which is smaller in size than, but substantially homologous to the polypeptide it is a fragment of, and where the functional fragment polypeptide sequence is about at least 50%, or 60% or 70% or at 80% or 90% or 100% or greater than 100%, for example 1.5-fold, 2-fold, 3-fold, 4-fold or greater than 4-fold effective biological action as the polypeptide from which it is a fragment of Functional fragment polypeptides may have additional functions that can include decreased antigenicity, increased DNA binding (as in transcription factors), or altered RNA binding (as in regulating RNA stability or degradation).

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

As used herein, the term “transcription factor” refers to a protein that binds to specific parts of DNA using DNA binding domains and is part of the system that controls the transfer (or transcription) of genetic information from DNA to RNA. As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, i.e., “selective conditions.” To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

A “reporter gene” as used herein encompasses any gene that is genetically introduced into a cell that adds to the phenotype of the stem cell. Reporter genes as disclosed in this invention are intended to encompass fluorescent, luminescent, enzymatic and resistance genes, but also other genes which can easily be detected by persons of ordinary skill in the art. In some embodiments of the invention, reporter genes are used as markers for the identification of particular stem cells, cardiovascular stem cells and their differentiated progeny. A reporter gene is generally operatively linked to sequences that regulate its expression in a manner dependent upon one or more conditions which are monitored by measuring expression of the reporter gene. In some cases, expression of the reporter gene may be determined in live cells. Where live cell reporter gene assays are used, reporter gene expression may be monitored at multiple time points, e.g., 2, 3, 4, 5, 6, 8, or 10 or more time points. In some cases, where a live cell reporter assay is used, reporter gene expression is monitored with a frequency of at least about 10 minutes to about 24 hours, e.g., 20 minutes, 1 hour, 2 hours, 3 hours, 4 hours, 5 hours, 6 hours, 7 hours, 8 hours, 9 hours, 10 hours, 12 hours, 18 hours, or another frequency from any integer between about 10 minutes to about 24 hours.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “test compound” refers to any of a small molecule, nucleic acid, amino acid, polypeptide, antibody and antibody-like molecules, aptamers, macrocycles, or other molecules. In certain embodiments, a test compound is a small organic molecule. In one aspect of these embodiments, the small organic molecule has a molecular weight of less than about 5,000 daltons. In certain embodiments, the test compound is other than an amino acid. In other embodiments, the small molecule is other than leucine, isoleucine or analogs of either of the foregoing.

As used herein, “neuro disorder” or “neuro disease” refer to neurodegenerative disorders, neuropsychiatric disorders and/or neurodevelopmental disorders. Neuro disoders may be any disease affecting neuronal network connectivity, synaptic function and activity. “Neurodegenerative disorder” refers to a disease condition involving neural loss mediated or characterized at least partially by at least one of deterioration of neural stem cells and/or progenitor cells. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral haemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or haemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Stem Cells

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

While certain embodiments are described below in reference to the use of stem cells for producing neural tissues or precursors thereof, germ cells may be used in place of, or with, the stem cells to provide at least one cerebral organoid, using similar protocols as the illustrative protocols described herein. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

ES cells, e.g., human embryonic stem cells (hESCs) or mouse embryonic stem cells (mESCs), with a virtually endless replication capacity and the potential to differentiate into most cell types, present, in principle, an unlimited starting material to generate the differentiated cells for clinical therapy (available on the world wide web at stemcells.nih.gov/info/scireport/2006report.htm, 2006).

hESC cells, are described, for example, by Cowan et al. (N Engl. J. Med. 350:1353, 2004) and Thomson et al. (Science 282:1145, 1998); embryonic stem cells from other primates, Rhesus stem cells (Thomson et al., Proc. Natl. Acad. Sci. USA 92:7844, 1995), marmoset stem cells (Thomson et al., Biol. Reprod. 55:254, 1996) and human embryonic germ (hEG) cells (Shamblott et al., Proc. Natl. Acad. Sci. USA 95:13726, 1998) may also be used in the methods disclosed herein. mESCs, are described, for example, by Tremml et al. (Curr Protoc Stem Cell Biol. Chapter 1:Unit 1C.4, 2008). The stem cells may be, for example, unipotent, totipotent, multipotent, or pluripotent. In some examples, any cells of primate origin that are capable of producing progeny that are derivatives of at least one germinal layer, or all three germinal layers, may be used in the methods disclosed herein.

In certain examples, ES cells may be isolated, for example, as described in Cowan et al. (N Engl. J. Med. 350:1353, 2004) and U.S. Pat. No. 5,843,780 and Thomson et al., Proc. Natl. Acad. Sci. USA 92:7844, 1995. For example, hESCs cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hESCs include their pluripotent derivatives, such as primitive ectoderm-like (EPL) cells, as outlined, for example, in WO 01/51610 (Bresagen). hESCs can also be obtained from human pre-implantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses can be isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers. After 9 to 15 days, inner cell mass-derived outgrowths can be dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology can be individually selected by micropipette, mechanically dissociated into clumps, and replated. ES-like morphology is characterized as compact colonies with apparently high nucleus to cytoplasm ratio and prominent nucleoli. Resulting hESCs can then be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some examples, clump sizes of about 50 to 100 cells are optimal. mESCs cells can be prepared from using the techniques described by e.g., Conner et al. (Curr. Prot. in Mol. Biol. Unit 23.4, 2003).

Embryonic stem cells can be isolated from blastocysts of members of the primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, as outlined in WO 01/51610 (Bresagen).

Alternatively, in some embodiments, hES cells can be obtained from human preimplantation embryos. Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage in G1.2 and G2.2 medium (Gardner et al., Fertil. Steril. 69:84, 1998). The zona pellucida is removed from developed blastocysts by brief exposure to pronase (Sigma). The inner cell masses are isolated by immunosurgery, in which blastocysts are exposed to a 1:50 dilution of rabbit anti-human spleen cell antiserum for 30 min, then washed for 5 min three times in DMEM, and exposed to a 1:5 dilution of Guinea pig complement (Gibco) for 3 min (Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps, either by exposure to calcium and magnesium-free phosphate-buffered saline (PBS) with 1 mM EDTA, by exposure to dispase or trypsin, or by mechanical dissociation with a micropipette; and then replated on mEF in fresh medium. Growing colonies having undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (containing 2 mM EDTA), exposure to type IV collagenase (^{˜}200 U/mL; Gibco) or by selection of individual colonies by micropipette. Clump sizes of about 50 to 100 cells are optimal.

In some embodiments, human Embryonic Germ (hEG) cells are pluripotent stem cells which can be used in the methods as disclosed herein to differentiate into primitive endoderm cells. hEG cells can be used be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622, which is incorporated herein in its entirety by reference.

Briefly, genital ridges processed to form disaggregated cells. EG growth medium is DMEM, 4500 mg/L D-glucose, 2200 mg/L mM NaHCO₃; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/mL human recombinant bFGF (Genzyme); and 10 μM forskolin (in 10% DMSO). Ninety-six well tissue culture plates are prepared with a sub-confluent layer of feeder cells (e.g., STO cells, ATCC No. CRL 1503) cultured for 3 days in modified EG growth medium free of LIF, bFGF or forskolin, inactivated with 5000 rad γ-irradiation ^{˜}0.2 mL of primary germ cell (PGC) suspension is added to each of the wells. The first passage is done after 7-10 days in EG growth medium, transferring each well to one well of a 24-well culture dish previously prepared with irradiated STO mouse fibroblasts. The cells are cultured with daily replacement of medium until cell morphology consistent with EG cells is observed, typically after 7-30 days or 1-4 passages.

In certain examples, the stem cells can be undifferentiated (e.g. a cell not committed to a specific linage) prior to exposure to at least one differentiation medium and/or agent according to the methods as disclosed herein, whereas in other examples it may be desirable to differentiate the stem cells to one or more intermediate cell types prior to exposure of the at least one differentiation medium or agent described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

ES cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subjects adipose tissue, for example using the CELUTION™ SYSTEM from Cytori, as disclosed in U.S. Pat. Nos. 7,390,484 and 7,429,488 which is incorporated herein in its entirety by reference.

In some embodiments, human umbilical cord blood cells (HUCBC) are useful in the methods as disclosed herein. Human UBC cells are recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497). Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79; 1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096).

In another embodiment, pluripotent cells are cells in the hematopoietic micro-environment, such as the circulating peripheral blood, preferably from the mononuclear fraction of peripheral blood, umbilical cord blood, bone marrow, fetal liver, or yolk sac of a mammal. The stem cells, especially neural stem cells, may also be derived from the central nervous system, including the meninges.

In another embodiment, pluripotent cells are present in embryoid bodies are formed by harvesting ES cells with brief protease digestion, and allowing small clumps of undifferentiated human ESCs to grow in suspension culture. Differentiation is induced by withdrawal of conditioned medium. The resulting embryoid bodies are plated onto semi-solid substrates. Formation of differentiated cells may be observed after around about 7 days to around about 4 weeks. Viable differentiating cells from in vitro cultures of stem cells are selected for by partially dissociating embryoid bodies or similar structures to provide cell aggregates. Aggregates comprising cells of interest are selected for phenotypic features using methods that substantially maintain the cell to cell contacts in the aggregate.

In an alternative embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

Cloning and Cell Culture

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, for example, in Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Scientists at Geron have discovered that pluripotent SCs can be maintained in an undifferentiated state even without feeder cells. The environment for feeder-free cultures includes a suitable culture substrate, particularly an extracellular matrix such as MATRIGEL® (gelatinous protein mixture) or laminin. Typically, enzymatic digestion is halted before cells become completely dispersed (say, .about.5 min with collagenase IV). Clumps of ^{˜}10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (^{˜}4,000 rad) primary mouse embryonic fibroblasts, telomerized mouse fibroblasts, or fibroblast-like cells derived from pPS cells. Medium can be conditioned by plating the feeders at a density of ^{˜}5-6×10⁴ cm⁻² in a serum free medium such as KO DMEM supplemented with 20% serum replacement and 4 ng/mL bFGF. Medium that has been conditioned for 1-2 days is supplemented with further bFGF, and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

Generating Functional Neurons

Aspects of the disclosure relate to generating functional neurons (e.g., cortical neurons, motor neurons, and the like). Generally, the functional neurons produced according to the methods disclosed herein demonstrate several hallmarks of mature, functional neurons, including, but not limited to, synapse formation, expression of functional receptors for neurotransmitters, spontaneous electrical activity, and appropriate expression of activity-induced genes.

The functional neurons can be produced according to any suitable culturing protocol to differentiate a stem cell or pluripotent cell to a desired stage of differentiation. In some embodiments, the functional neurons are produced by culturing at least one stem cell for a period of time and under conditions suitable for the at least one stem cell to differentiate into the neural tissue or a precursor thereof.

In some embodiments, the functional neurons or precursor thereof is a substantially pure population of functional neurons or precursors thereof. In some embodiments, a population of functional neurons or precursors thereof comprises a mixture of pluripotent cells or differentiated cells. In some embodiments, a population of functional neurons or precursors thereof is substantially free or devoid of embryonic stem cells or pluripotent cells.

In some embodiments, the functional neurons or precursors thereof are maintained in culture by methods known by one of ordinary skill in the art, and in some embodiments, propagated prior to being converted into functional neurons by the methods as disclosed herein.

In certain embodiments, stem cells (e.g., hESCs or iPSCs) are maintained as undifferentiated pluripotent spheres in spin culture. The stem cells may be grown in mTeSR medium. In some embodiments, neutralization is initiated by supplementing the mTESR medium with an activin/TGF-β inhibitor and a BMP inhibitor. In some aspects, the activin/TGF-β inhibitor is SB431542. In some aspects, the BMP inhibitor is LDN193189. The combination of the activin/TGF-β inhibitor and BMP inhibitor may be referred to as dual SMAD inhibition.

In certain embodiments, the stem cells may be differentiated to form cortical neurons. In certain aspects, the pluripotent spheres were gradually adapted to neural induction medium (NIM). The pluripotent spheres may be gradually adapted to NIM through a dilution series of KSR and NIM. In certain aspects, the dilution series of KSR and NIM includes dual SMAD inhibition. In some aspects, KSR media includes a supplemental agent. The supplemental agent may include a Wnt signaling inhibitor. In certain embodiments, the Wnt signaling inhibitor is XAV939. In some embodiments, the dilution series of KSR and NIM media may include 75% KSR and 25% NIM, 50% KSR and 50% NIM, and 25% KSR and NIM 75% media. In some aspects, the dilution series occurs over a period of 1, 2, 3, 4, 5, 6, or 7 days. In certain embodiments, the spheres subjected to differentiation may be maintained in NIM. The spheres subjected to differentiation may be maintained in NIM for a period of 8 to 15 days, and in certain embodiments for a period of 10 days. In certain embodiments, differentiated spheres may be maintained in nuerobasal medium. In certain embodiments, differentiated spheres may be maintained in nuerobasal medium for a period of 1 to 150, 15 to 130, or 30 to 120 days.

In certain embodiments, the stem cells may be differentiated to form motor neurons. In certain aspects, the pluripotent spheres were gradually adapted to neural induction medium (NIM). The pluripotent spheres may be gradually adapted to NIM through a dilution series of KSR and NIM. In certain aspects, the dilution series of KSR and NIM includes dual SMAD inhibition. In some aspects, KSR media includes a supplemental agent. The supplemental agent may include retinoic acid (RA), brain-derived neurotrophic factor (BDNF), and/or smoothened agonist (SAG). In some embodiments, the dilution series of KSR and NIM media may include 100% KSR media; 100% KSR media supplemented with RA; 75% KSR media and 25% NIM supplemented with RA and BDNF; 50% KSR and 50% NIM supplemented with RA, SAG, and BDNF; 25% KSR and 75% NIM supplemented with RA, SAG and BDNF. In some aspects, the dilution series occurs over a period of 1, 2, 3, 4, 5, 6, 7, 8, 9, or 10 days. In certain embodiments, the spheres subjected to differentiation may be maintained in NIM. In some embodiments, the spheres subjected to differentiation are maintained in NIM supplemented with RA, SAG, BDNF, and DAPT. The spheres subjected to differentiation may be maintained supplemented NIM for a period of 3 to 8 days, and in certain embodiments for a period of 5 days. In certain embodiments, differentiated spheres may be maintained in NB medium. The NB medium may include a supplemental agent. The supplemental agent may include BDNF, Glial cell-derived neurotrophic factor (GDNF) and/or Ciliary neurotrophic factor (CNTF). In certain embodiments, differentiated spheres may be maintained in supplemented NB medium for a period of 1 to 150, 15 to 130, or 30 to 120 days.

Alternative detailed protocols for generating neural tissue, include, but are not limited to, those described in Lancaster, et al. Nature Protocols, 9.10:2329 (2014), U.S. Patent Application No. 62/273,795 (Docket No. HRVY-077-001, filed Dec. 31, 2015; inventors Arlotta, et al.), and U.S. Application No. 62/342,566 (Docket No. HRVY-077-002, filed May 27, 2016; inventors Arlotta, et al.), which are incorporated herein by reference. In some embodiments, the detailed protocols may be modified.

Functional Neurons

In some embodiments, the disclosure provides functional neurons. In some aspects, the functional neurons include functional cortical neurons. In other aspects, the functional neurons include functional motor neurons.

In some embodiments, the neurons that are or resemble functional neurons (e.g., cortical neurons or motor neurons) comprise substantially all cells found in the brain or progenitors thereof. One can use any means common to one of ordinary skill in the art to confirm the presence of functional neurons produced by the differentiation of stem cells or precursors thereof by exposure to at least one differentiation medium or/or supplemental agent as described herein. In some embodiments, such neurons can be identified by selective gene expression markers. In some embodiments, the method can include detecting the positive expression (e.g. the presence) of a marker for neurons. In some embodiments, the marker can be detected using a reagent. A reagent for a marker can be, for example, an antibody against the marker or primers for a RT-PCR or PCR reaction, e.g., a semi-quantitative or quantitative RT-PCR or PCR reaction. Such markers can be used to evaluate whether a neuron has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of at least one stem cell or precursor thereof to a functional neuron can be monitored by determining the expression of markers characteristic of neurons (e.g., cortical neurons or motor neurons). In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of functional neurons, as well as the lack of significant expression of markers characteristic of the stem cells or precursors thereof is determined.

As described in connection with monitoring the production of neural tissue from a stem cell, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as quantitative-PCR by methods ordinarily known in the art. Additionally, it will be appreciated that at the polypeptide level, many of the markers of pancreatic islet hormone-expressing cells are secreted proteins. As such, techniques for measuring extracellular marker content, such as ELISA, may be utilized.

In some embodiments, cells of the inventive culture express one or more gene expression markers selected from forebrain markers BF1 and Six3. Alternatively, or in addition, cells of the inventive culture express one or more gene expression markers selected from hindbrain markers Krox20 and Ils1. At a certain stage of development forebrain markers are expressed in increased amounts as compared to hindbrain markers in the tissue.

In some embodiments, the neuron tissue culture can alternatively or in addition be characterized by comprising cells expressing one or more markers selected from Otx1, Otx2, FoxG1, Auts2, Tbr2, Tuj 1, Brn2, Satb2, Ctip2, calretinin, or any combination thereof. In some embodiments, the neuron tissue culture can be characterized by comprising cells expressing one or more markers selected from Pax6, Sox2, Phospho-vimentin, Tbr2, Tuj 1, DCX, Foxg1, Ttr, Prox1, Fzd9, Nkx2.1, Reelin, Tbr1, Ctip2, Satb2, TH, opsin, s100beta or any combination thereof. These markers may be expressed during any stage of the culture during the described methods, and may be expressed in the provided tissue culture.

In certain embodiments, the neuron tissue culture comprises cells, which express Otx1 and/or Oxt2. Otx1 and/or Oxt2 are expressed in cells of forebrain/midbrain identity. Otx1 and/or Otx2 are neural progenitor genes. In certain embodiments, the neuron tissue culture comprises cells, which express FoxG1. FoxG1 is expressed in cells of telencephalic identity. FoxG1 is an established marker of cortical progenitor cells. In certain embodiments, the neuron tissue culture comprises cells, which express Auts2. Auts2 is expressed in cells of frontal cortex identity. In certain embodiments, the neuron tissue culture comprises cells, which express Tuj 1. Tuj 1 is expressed in cells of a cortical inner fiber layer identity. In certain embodiments, the neuron tissue culture comprises cells, which express Brn2. Brn2 is expressed in a number of neuronal subtypes including cells localized in the upper layers of the cerebral cortex. In certain embodiments, the neuron tissue culture comprises cells, which express Satb2. Satb2 is expressed in a number of neuronal subtypes including callosal projection neurons localized in the upper layers of the cerebral cortex. In certain embodiments, the neuron tissue culture comprises cells, which express Ctip2. Ctip2 is expressed in a number of neuronal subtypes including cells localized in the deep layers of the cerebral cortex. In certain embodiments, the neuron tissue culture comprises cells, which express calretinin. Calretinin is expressed in a number of neuronal subtypes including GABAeric cortical interneurons.

In certain embodiments, the neuron tissue culture comprises cells, which express Pax6. Pax6 is expressed in radial glia/NSCs cells. In certain embodiments, the neuron tissue culture comprises cells, which express Sox2. Sox2 is expressed in radial glia/NSCs cells. In certain embodiments, the neuron tissue culture comprises cells, which express Phospho-vimentin. Phospho-vimentin is expressed in radial glia/NSCs cells. In certain embodiments, the neuron tissue culture comprises cells, which express Tbr2. Tbr2 is expressed in intermediate cortical progenitor cells. In certain embodiments, the neuron tissue culture comprises cells, which express Tuj 1. Tuj 1 is expressed in neuron cells. In certain embodiments, the neuron tissue culture comprises cells, which express DCX. DCX is expressed in differentiating neuronal cells. In certain embodiments, the neuron tissue culture comprises cells, which express Foxg1. Foxg1 is expressed in forebrain cells. In certain embodiments, the neuron tissue culture comprises cells, which express Ttr. Ttr is expressed in choroid plexus cells. In certain embodiments, the neuron tissue culture comprises cells, which express Prox1. Prox1 is expressed in a number of neuronal subtypes including hippocampal neurons. In certain embodiments, the neuron tissue culture comprises cells, which express Fzd9. Fzd9 is expressed in a number of neuronal subtypes including hippocampal neurons. In certain embodiments, the neuron tissue culture comprises cells, which express Nkx2.1. Nkx2.1 is expressed in ventral forebrain cells. In certain embodiments, the neuron tissue culture comprises cells, which express Reelin. Reelin is expressed in a number of neuronal subtypes including Cajal-retzius cells. In certain embodiments, the neuron tissue culture comprises cells, which express Tbr1. Tbr1 is expressed in a number of neuronal subtypes including deep-layer cortical neurons. In certain embodiments, the neuron tissue culture comprises cells, which express Ctip2. Ctip2 is expressed in deep-layer cortical neurons. In certain embodiments, the neuron tissue culture comprises cells, which express Satb2. Satb2 is expressed in surface-layer neuron cells. In certain embodiments, the neuron tissue culture comprises cells, which express Cux1. Cux1 is expressed in a number of neuronal subtypes including cells localized in the upper layers of the cerebral cortex. In certain embodiments, the neuron tissue culture comprises cells, which express the marker opsin. Opsin is expressed in a number of neuronal subtypes including photoreceptors localized in the retina. In certain embodiments, the neuron tissue culture comprises cells, which express TH. TH is a marker of dopaminergic neurons. In certain embodiments, the neuron tissue culture comprises cells, which express s100 beta. s100 beta is expressed in glial cells including astrocytes.

In certain embodiments, the neuron tissue culture comprises cells, which express ISL1. ISL1 is expressed in motor neuron cells. In certain embodiments, the neuron tissue culture comprises cells, which express HB9. HB9 is expressed in motor neuron cells. In certain embodiments, the neuron tissue culture comprises cells, which express ChAT. ChAT is expressed in motor neuron cells. In certain embodiments, the neuron tissue culture comprises cells, which express MAP2. MAP2 is expressed in motor neuron cells.

It is understood that the present invention is not limited to those markers listed as functional neuron markers herein, and the present invention also encompasses markers such as cell surface markers, antigens, and other gene products including ESTs, RNA (including microRNAs and antisense RNA), DNA (including genes and cDNAs), and portions thereof.

In some embodiments, the methods of the invention allow for the generation of functional neurons that exhibit one or more features. In certain embodiments, the one or more features include forming synapses with neighboring cells, exhibit spontaneous electrical activity, and respond to depolarization. In certain aspects, the cerebral organoids exhibit a long survival time (e.g., maintained for up to 4-5 months).

In some embodiments, the methods of the invention allow for the generation of functional cortical neurons. The neurons may maintain expression of cortical markers, and in some aspects, express pre- and post-synaptic proteins. In some aspects, the cortical neurons may form synaptic connections and neural networks. In certain embodiments, cortical neurons from dissociated cortical spheres exhibit voltage-gated Na⁺ and K⁺ currents, and in certain aspects exhibited fire action potentials upon current injection. Cortical neurons may exhibit spontaneous synaptic transmission, thereby demonstrating that the cells are functional, electrophysiologically active cells. In certain embodiments, the cortical neurons display voltage-gated Na⁺ and K⁺ currents, intracellular Ca²⁺ dynamics, and activity-induced gene expression characteristics of cortical neurons.

In some embodiments, the methods of the invention allow for the generation of functional motor neurons. The methods of the present invention allow for the generation of a very efficient yield of motor neurons (e.g., up to 85%). Motor neurons derived from spin culture spheres survived for an extended period of time in culture (e.g., 4 to 5 months). In certain embodiments, the motor neurons exhibit significant levels of activity, with increasing fluctuations in fluorescence over time. In some aspects, groups of motor neurons exhibit synchronized Ca²⁺ dynamics. In certain embodiments, motor neurons from dissociated motor spheres exhibit voltage-gated Na⁺ and K⁺ currents, and in certain aspects exhibited fire action potentials upon current injection. Motor neurons may exhibit spontaneous synaptic transmission. In certain aspects, the motor neurons are in contact with muscle fibers and form neuromuscular junctions.

Screening of Diseases

The invention provides a method of screening patients with neurodevelopmental, neurodegenerative and neuropsychiatric disease, and more broadly all diseases affecting synaptic function, neuronal network activity and stimulation, through the generation of functional neurons from patient derived induced pluripotent stem cells (iPSCs). Functional neurons can also be generated from iPSCs that are genetically engineered to carry mutations associated with neurodevelopmental, neurodegenerative and neuropsychiatric disease. A subject or patient can be one who has been previously diagnosed with or identified as suffering from or having a condition, disease, or neuropsychiatric disorder described herein in need of treatment or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. Rather, a subject can include one who exhibits one or more risk factors or symptoms for a condition or one or more complications related to a condition. A “subject in need” of treatment for a particular condition can be a subject having that condition, diagnosed as having that condition, or at increased risk of developing that condition relative to a given reference population.

In some embodiments, the methods described herein comprise selecting a subject diagnosed with, suspected of having, or at risk of developing a neuropsychiatric disorder as described herein.

In some aspects, patent derived neural tissue is screened for a neurodevelopmental, neurodegenerative and neuropsychiatric disease and/or for pathology of neuronal network connectivity, synaptic function and activity. In some aspects, the disease may be depression, obsessive-compulsive disorder, schizophrenia, visual hallucination, auditory hallucination, eating disorder, bipolar disorder, epilepsy, autism, ALS and any disease affecting of neuronal network connectivity, synaptic function and activity.

The disclosure contemplates methods in which functional neurons are generated according to the methods described herein from iPS cells derived from cells extracted or isolated from individuals suffering from a disease (e.g., epilepsy, autism, schizophrenia, bipolar disorder), and that generated functional neurons are compared to normal functional neurons from healthy individuals not having the disease to identify differences between the generated functional neurons and normal functional neurons which could be useful as markers for disease (e.g., neuropsychiatric, neurodegenerative, neurodevelopmental, and the like). In some embodiments, the iPS cells and/or functional neurons derived from patients are used to screen for agents (e.g., agents which are able to modulate genes contributing to a neuropsychiatric, neurodegenerative, or neurodevelopmental phenotype).

Screening of Agents to Treat a Neuropsychiatric, Neurodegenerative or Neurodevelopmental Disease

In some embodiments, the invention provides a method of screening test agents to identify treatment agents for a disease (e.g., a neurodegenerative, neuropsychiatric or neurodevelopmental disease). In some aspects, functional neurons exhibiting features of a disease are generated as described by the methods of the invention. The functional neurons may be generated from iPSCs obtained from a patient having a neurodegenerative disease or a neuropsychiatric disease (e.g., epilepsy, autism, bipolar disorder, schizophrenia). In certain embodiments, the functional neurons are treated with a test agent. A sensory stimulus may be applied to the treated neurons and the results measured and recorded. The stimulus results of the treated neurons may be compared to control levels. In certain embodiments, stimulus results of the treated neurons are similar to the control levels, exhibiting the beneficial effects of the test agent on a disorder. In alternative embodiments, stimulus results of the treated neurons are significantly different from the control levels, demonstrating that the test agent does not treat the specific disorder tested.

Compositions and Kits

Described herein are kits for practicing methods disclosed herein and for making functional neurons disclosed herein. In one aspect, a kit includes at least one stem cell or precursor thereof and at least one differentiation medium and/or supplemental agent as described herein, and optionally, the kit can further comprise instructions for converting at least one stem cell or precursor thereof to a population of functional neurons using a method described herein. In some embodiments, the kit comprises at least two differentiation mediums. In some embodiments, the kit comprises at least three differentiation mediums. In some embodiments, the kit comprises at least four differentiation mediums. In some embodiments, the kit comprises at least five differentiation mediums. In some embodiments, the kit comprises at least one supplemental agent. In some embodiments, the kit comprises at least two supplemental agents. In some embodiments, the kit comprises at least three supplemental agents. In some embodiments, the kit comprises at least four supplemental agents. In some embodiments, the kit comprises at least five supplemental agents. In some embodiments, the kit comprises differentiation mediums and/or supplemental agents for differentiating pluripotent cells to functional neurons.

In some embodiments, the kit comprises any combination of differentiation mediums and/or supplemental agents, e.g., for differentiating stem cells to functional neurons.

In one embodiment, the kit can comprise a pluripotent stem cell for the purposes of being used as a positive control, for example to assess or monitor the effectiveness or ability of a compound of formula (I) to chemically induce the pluripotent stem cell to differentiate into at least one functional neuron or precursors thereof. Accordingly, the kit can comprise sufficient amount of at least one differentiation medium and/or supplemental agent for inducing the differentiation of a control pluripotent stem cell population (positive control) as well as inducing the differentiation of a population of pluripotent stem cells of interest (e.g. the users preferred pluripotent stem cell e.g. an iPS cell) into at least one functional neuron.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of reactions e.g., 1, 2, 3 or greater number of separate reactions to induce pluripotent stem cells to functional neurons. A differentiation medium and/or supplemental agent can be provided in any form, e.g., liquid, dried or lyophilized form. It is preferred that a compound(s) (e.g., differentiation medium or agent) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a compound(s) (e.g., a differentiation medium and/or supplemental agent) as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing pluripotent stem cells (e.g., in vitro) or for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A differentiation medium and/or supplemental agent as described herein can be provided in any form, e.g., liquid, dried or lyophilized form. It is preferred that a compound(s) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing at least one differentiation medium and/or supplemental agent as described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The kit can also include a component for the detection of a marker for functional neurons, e.g., for a marker described herein, e.g., a reagent for the detection of functional neurons. Or in some embodiments, the kit can also comprise reagents for the detection of negative markers of functional neurons for the purposes of negative selection of functional neurons or for identification of cells which do not express these negative markers (e.g., functional neurons). The reagents can be, for example, an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

It may be desirable to perform an analysis of the karyotype of the functional neurons. Accordingly, the kit can include a component for karyotyping, e.g., a probe, a dye, a substrate, an enzyme, an antibody or other useful reagents for preparing a karyotype from a cell.

The kit can include functional neurons, e.g., functional neurons derived from the same type of stem cell or precursor thereof, for example for the use as a positive cell type control.

The kit can also include informational materials, e.g., instructions, for use of two or more of the components included in the kit.

The informational material can be descriptive, instructional, marketing or other material that relates to the methods described herein and/or the use of a compound(s) described herein for differentiating a pluripotent stem cell according to the methods described herein. In one embodiment, the informational material can include information about production of the compound, molecular weight of the compound, concentration, date of expiration, batch or production site information, and so forth. In one embodiment, the informational material relates to methods for culturing a population of stem cells in the presence of at least one differentiation medium and/or supplemental agent described herein.

### EXAMPLES

### Example 1—Spin Culture hPSCs Form Spheres that Adopt a Cortical Identity after Dual SMAD Inhibition

It has previously been shown that hPSCs can be cultured in three-dimensional (3D) spheres, consisting of hundreds of individual hPSCs, in a suspension-based system that can be readily scaled to permit the production of hundreds of millions of cells per culture (Ismadi et al., 2014; Otsuji et al., 2014; Pagliuca et al., 2014). Moreover, recent work has shown that hPSCs grown under such conditions can be differentiated into pancreatic beta cells that were more mature than those produced in standard two dimensional cultures (Pagliuca et al., 2014).

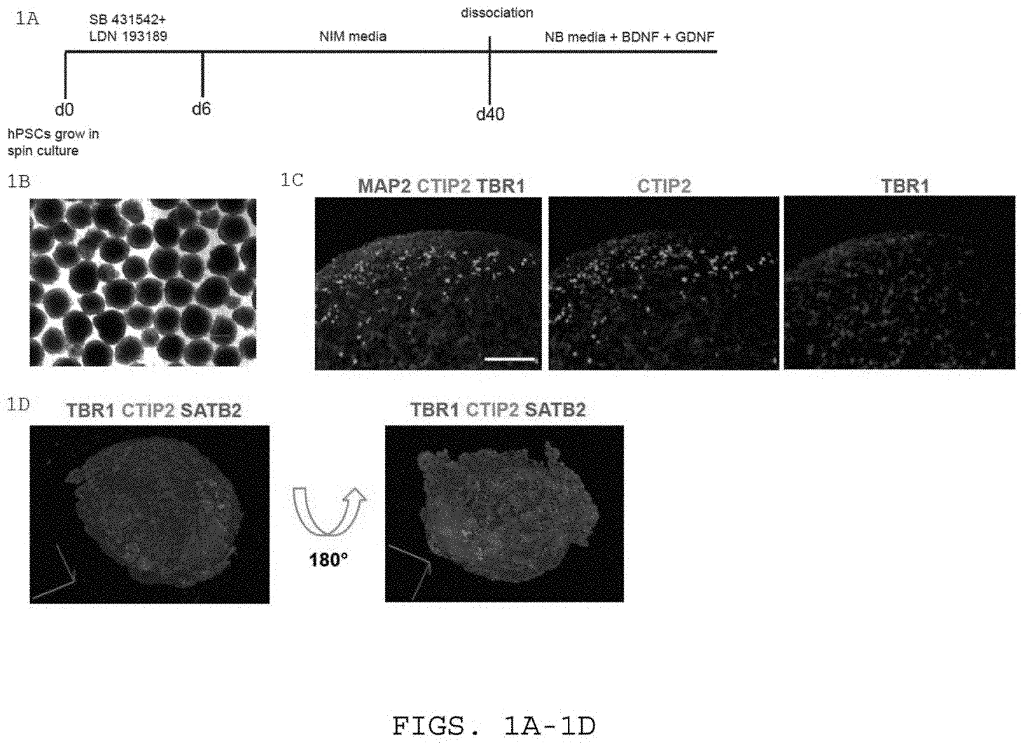

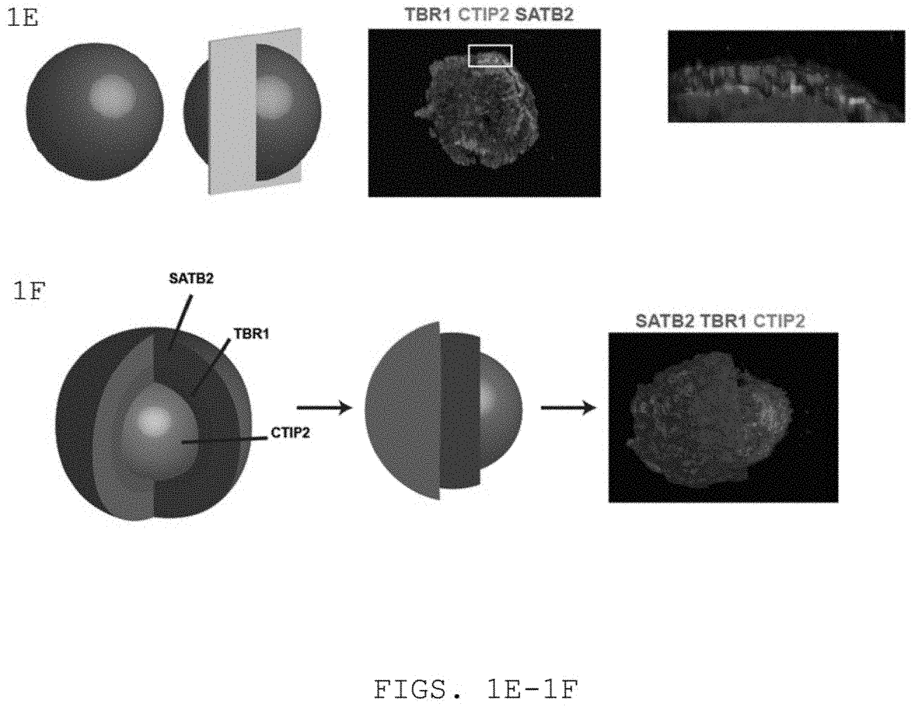

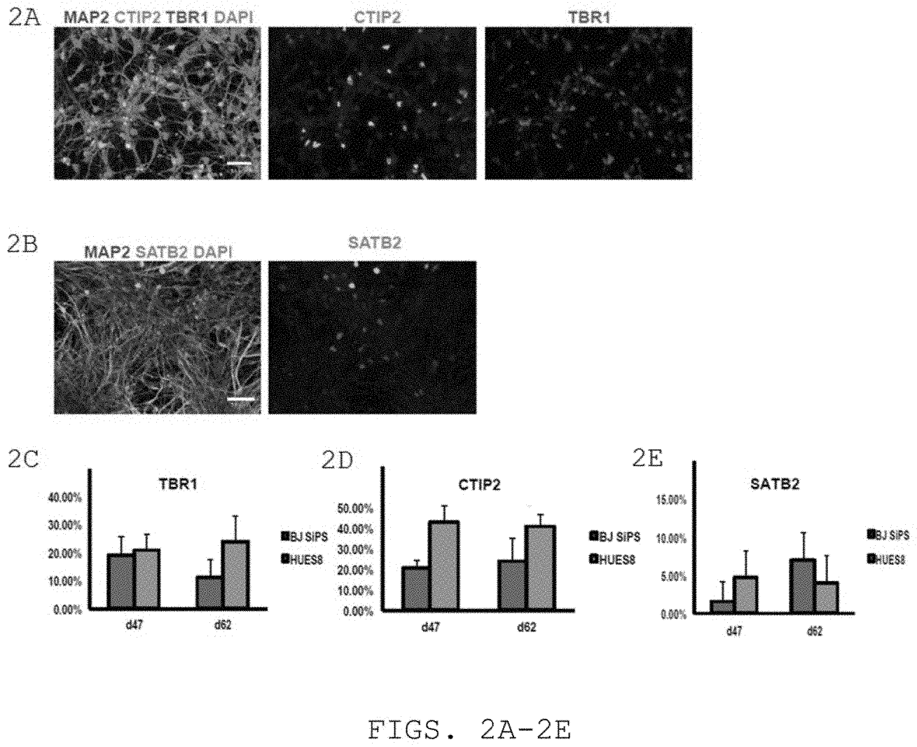

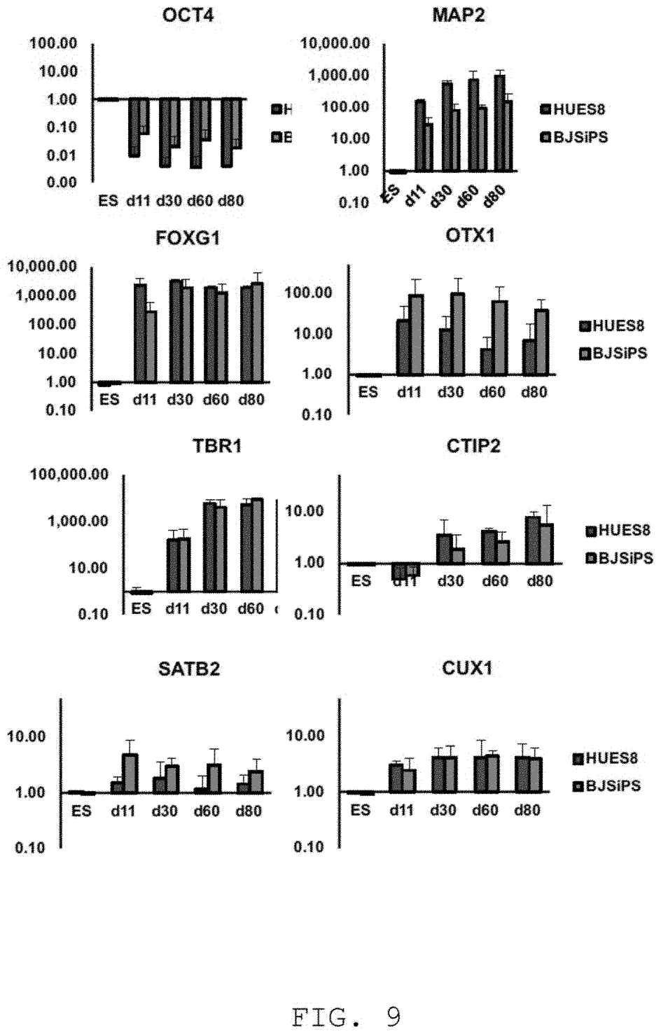

To explore the potential for generating cortical neurons using suspension-based cultures, we initially employed a differentiation protocol based on inhibition of the activin/TGFβ and BMP signaling pathways (Chambers et al., 2009) (illustrated schematically in FIG. 1A). Prior to differentiation, the size and general appearance of hPSC spheres was quite homogenous, and cells expressed the pluripotency markers OCT4 and NANOG (FIGS. 8A and 8B). During differentiation, spheres remained homogenous in size (FIG. 1B), and qRT-PCR analysis of RNA extracted from cells collected at different time points showed a rapid loss of expression of the pluripotency genes OCT4 and NANOG, and gradual expression of cortical progenitor, and the neuronal genes (FIG. 9). The neural progenitor genes OTX1 and OTX2, and FOXG1, an established marker of cortical progenitor cells, reached maximum levels of expression around day 11 of differentiation, while the temporal expression profile of genes specific for mature cortical layers seemed to resemble the sequence seen during embryonic development, with TBR1 (a specific marker of neurons in cortical layer VI) appearing first, CTIP2 (layer V) thereafter, and SATB2 and CUX1 (layers II, III, and IV) at later time points (FIG. 9).

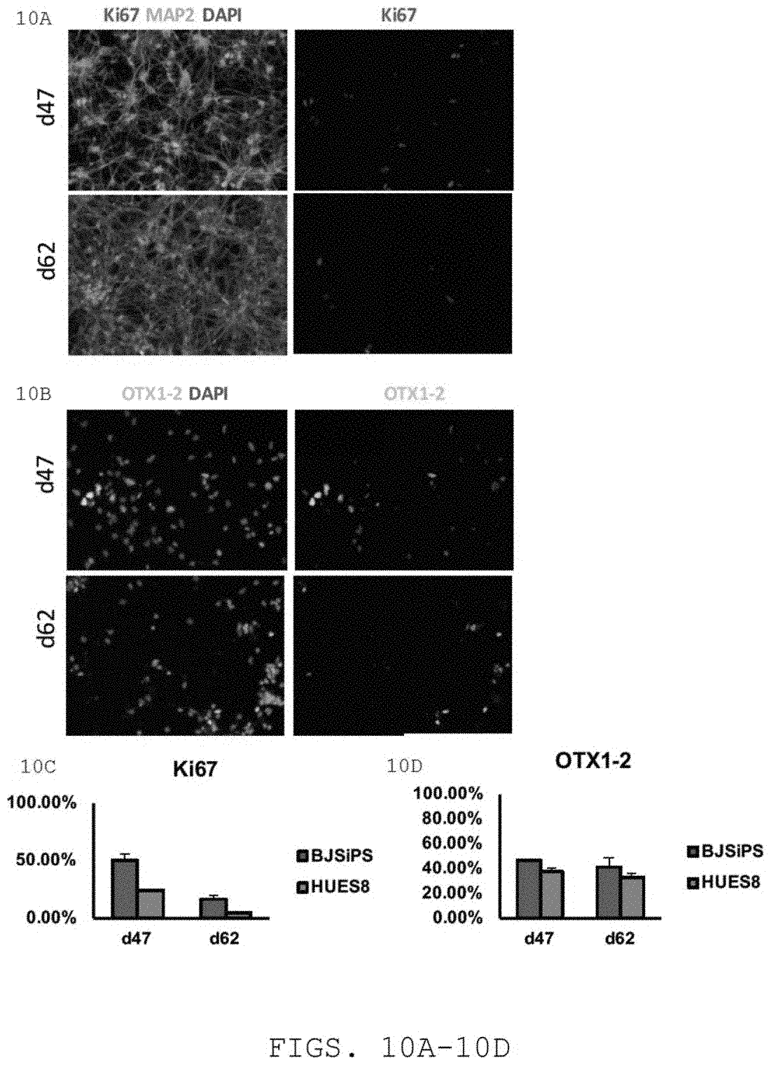

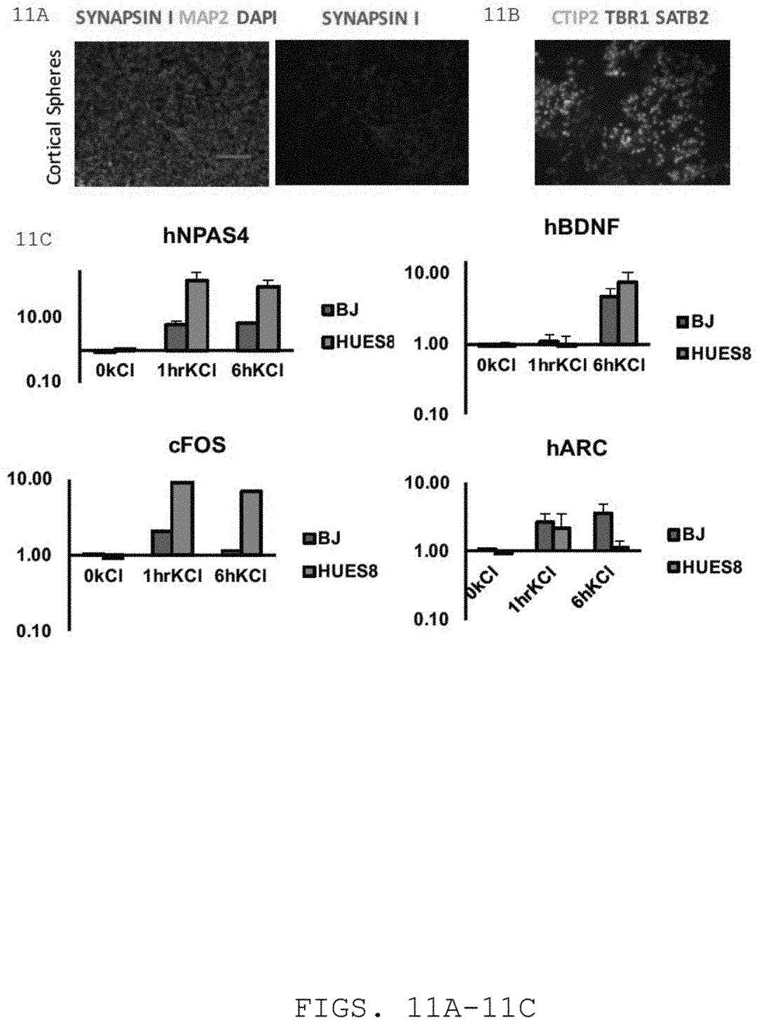

At day 50 of differentiation, spheres were collected and processed for immunocytochemistry using antibodies for cortical and synaptic markers. The results showed that spheres express the pan-neuronal marker MAP2, cortical neuron markers TBR1, CTIP2 and SATB2, and the synaptic marker SYNAPSIN1 (FIG. 1C and FIGS. 10A and 10B). Immunocytochemistry performed on sections of spheres indicated that neurons positive for markers of the different cortical layers were not arranged randomly, but rather appeared in clusters of cells expressing markers of particular cortical layers. To further substantiate this notion, we performed 3D light sheet fluorescence microscopy (LSFM) after clearing the spheres using the SeeDB method (Ke et al., 2013), allowing us to obtain a three-dimensional image of stained spheres. The data confirmed that neurons expressing markers or similar cortical layer identities were organized in clusters rather than being mixed in a random, stochastic manner (FIGS. 1D-1F and FIG. 8C).

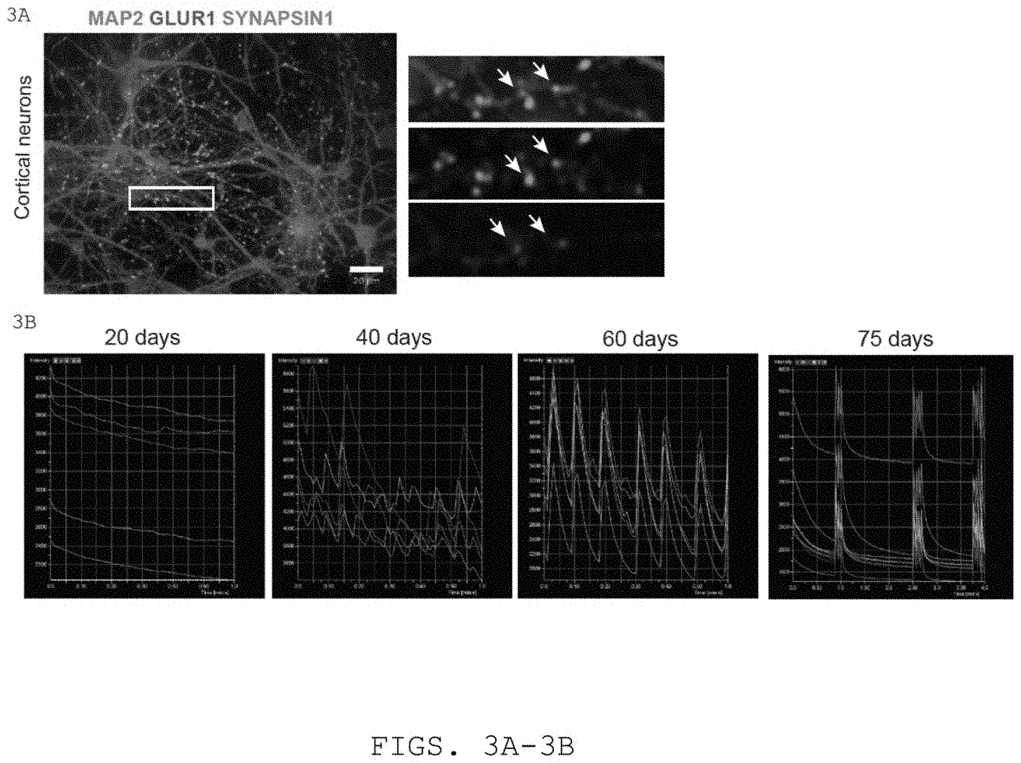

To obtain results with better cellular resolution to more readily quantify the efficiency and kinetics of neuronal differentiation, spheres differentiated for 40 days were dissociated, plated, and cultured for one additional week. Immunocytochemistry revealed the presence of neurons expressing the cortical markers TBR1, CTIP2, and SATB2 (FIGS. 2A-2B). Unbiased quantification revealed that cultures with up to 70% of all cells expressing one of these three markers could be obtained (FIGS. 2E-2E and Table 1). The remaining cells were positive for the neural progenitor markers OTX1 and 2 and the proliferation marker Ki67, consistent with the notion that these cells are cortical progenitors that have not differentiated to post mitotic cortical neurons. Furthermore, we found that the number of Ki67-positive cells declined with increased time in culture, suggesting that the progenitors would ultimately be depleted from the culture (FIG. 10). We did not detect any cells stained using an antibody raised against the glial marker GFAP, arguing against the presence of any astroglia in the spheres. Importantly, the spin culture protocol facilitates the generation of large numbers of neurons. From one spinner flask, we routinely obtained 3×10⁹ neurons. This spin-based method allows these cells to thrive for months without any signs of having necrotic cores.

These results show that hPSC spheres in spinner flasks differentiate efficiently to neuronal subtypes with neurons acquiring a predominantly cortical identity in the absence of patterning factors. Cortical cells expressing markers of different layers were readily identified and clustered together within the three-dimensional spheres, with CTIP2-expressing neurons predominantly residing centrally, SATB2-expressing neurons on the outside of the spheres, and TBR1-positive neurons sandwiched in between the other two populations. When dissociated and cultured as a monolayer, the neurons maintained expression of cortical markers.

TABLE 1
Quantification of the relative proportion of cortical subtypes
obtained from three independent experiments in two different hPSC
lines. Cells were dissociated at day 40, plated and processed for
immunocytochemistry with the indicated antibodies 7 (top) and
22 days (bottom) after dissociation. For each biological replicate
the total number of nuclei quantified is reported.
Cortical Immunostaining d 47

| d 47 | Tot Nuclei | % TBR1 | % CTIP2 | % SATB2 |
|---|---|---|---|---|
| BJ_1 | 21087 | 12% | 17% | 1% |
| BJ_2 | 4766 | 19% | 24% | 0.70% |
| BJ_3 | 10433 | 26% | 21% | 3% |
| Average | | 19% | 21% | 1.60% |
| STDEV | | 7% | 4% | 2.5% |
| HUES8_1 | 7483 | 17% | 36% | 1.20% |
| HUES8_2 | 27005 | 27% | 40% | 5% |
| HUES8_3 | 58148 | 18% | 52% | 8% |
| Average | | 21% | 43% | 4.73% |
| STDEV | | 5.51% | 8.33% | 3.41% |

Cortical Immunostaining d 62

| d 62 | Tot Nuclei | % TBR1 | % CTIP2 | % SATB2 |
|---|---|---|---|---|
| BJ_1 | 27331 | 3% | 23% | 3% |
| BJ_2 | 7525 | 14% | 35% | 10% |
| BJ_3 | 7017 | 15% | 13% | 8% |
| Average | | 11% | 24% | 7% |
| STDEV | | 7% | 11% | 4% |
| HUES8_1 | 8417 | 23% | 48% | 1% |
| HUES8_2 | 47898.00 | 16% | 38% | 8% |
| HUES8_3 | 50666.00 | 34% | 38% | 4% |
| Average | | 24% | 41% | 4% |
| STDEV | | 9.07% | 5.77% | 3.51% |

### Example 2—Dissociated Cortical Neurons from Spontaneously Active Neuronal Networks and Exhibit Stimulation-Induced Activity

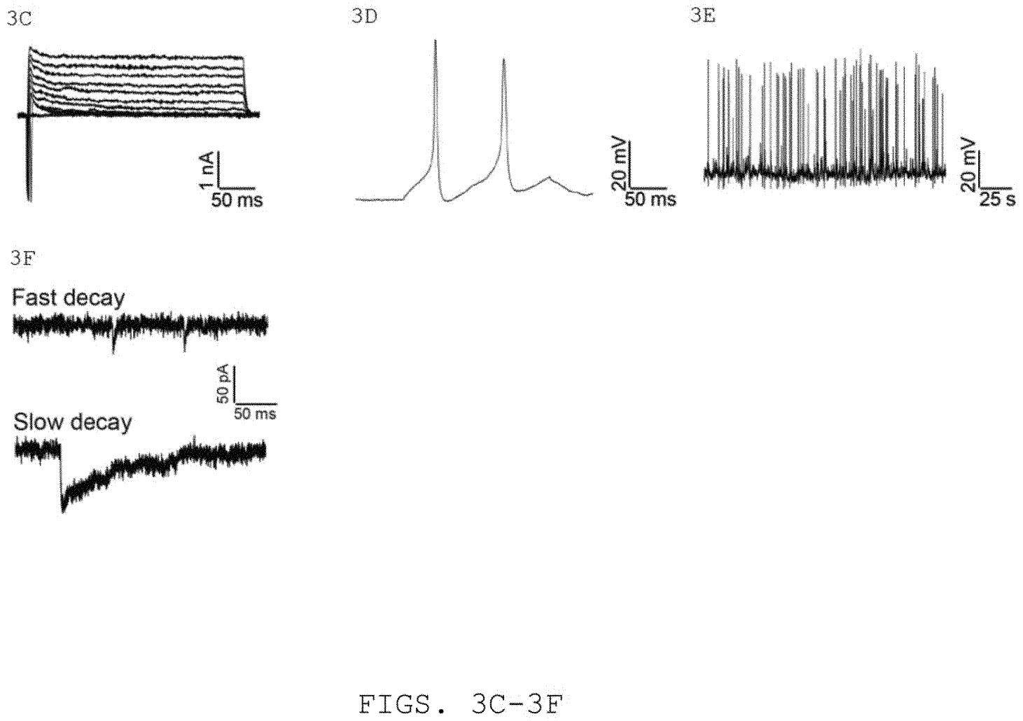

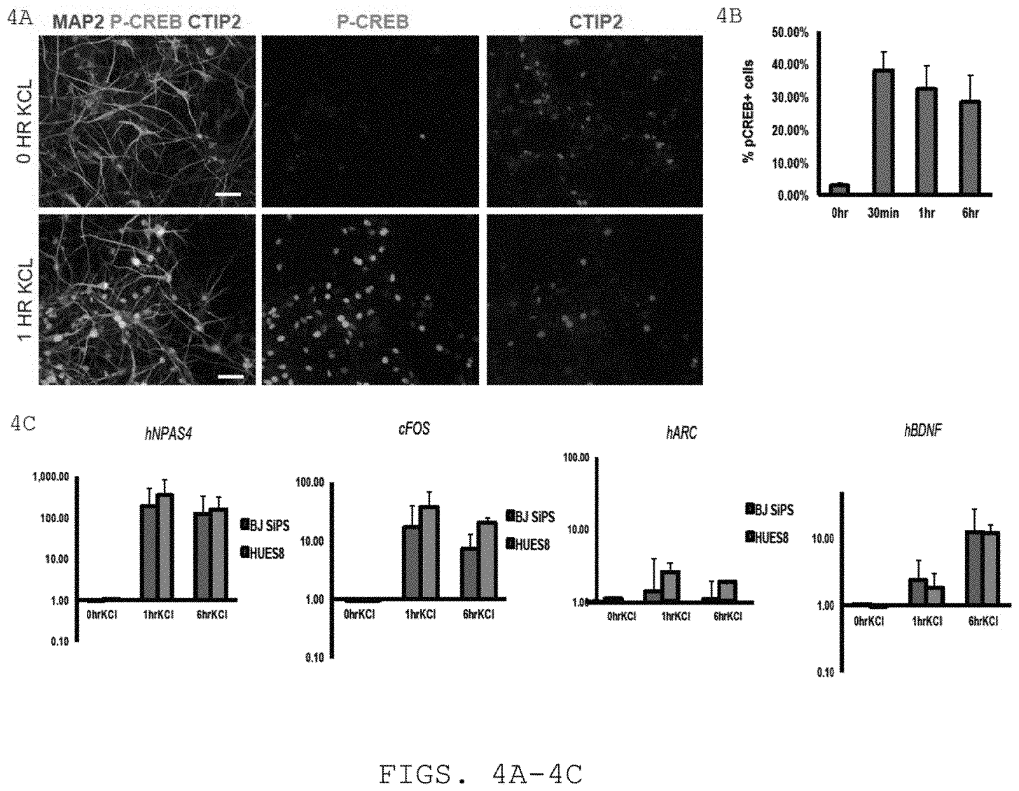

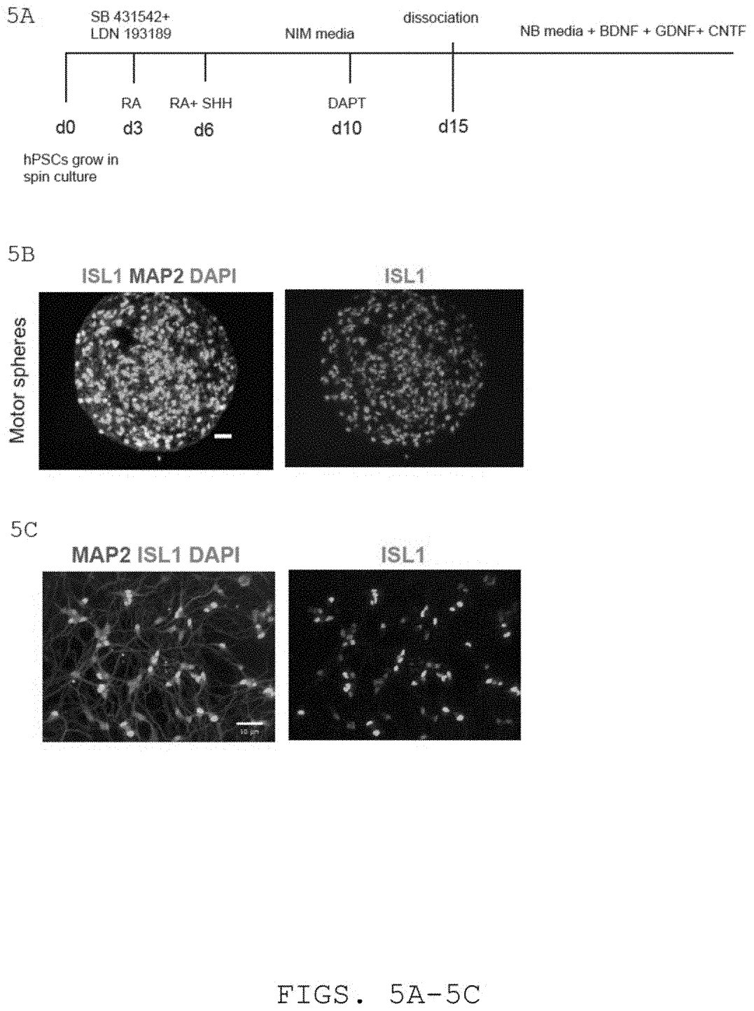

We thereafter sought to further characterize dissociated post-mitotic neurons. We analyzed these neurons for synaptic markers using antibodies specific for MAP2, the pre-synaptic marker SYNAPSIN1, and the post-synaptic marker GLUR1, specific for glutamatergic synapses. The results showed the presence of distinct SYNAPSIN1- and GLUR1-immunoreactive puncta distributed along the MAP2-positive dendrites. SYNAPSIN1 and GLUR1 immunoreactivity was not completely overlapping, but immunoreactive puncta were frequently found in close apposition, suggesting the formation of synaptic connections (FIG. 3A).

In an attempt to address whether hPSC-derived cortical neurons exhibit spontaneous electrical activity in culture, we first monitored intracellular Ca²⁺ dynamics as a surrogate for electrical activity by performing time-lapse microscopy on cultures of dissociated neurons transduced with adeno-associated virus (AAV) expressing the genetically encoded Ca²⁺ sensor GCaMP6 (Chen et al., 2013). Cells were transduced three days after dissociation and imaged every 10 days. Early after dissociation, neurons exhibited low levels of activity while fluctuations in fluorescence increased over time, and all cultures of cortical neurons exhibited synchronized Ca²⁺ waves 60 days after dissociation (FIG. 3B). In order to further examine the electrophysiological characteristics of neurons from dissociated cortical spheres we conducted patch-clamp recordings. After six weeks in culture, cortical neurons showed typical voltage-gated Na⁺ and K⁺ currents (FIG. 3C) and were able to fire action potentials upon current injection (FIG. 3D). Spontaneous firing of action potentials and postsynaptic currents (FIGS. 3E and 3F) was also observed in most cells recorded (4/5), showing that cortical neurons obtained from dissociated spheres are able to mature into functional, electrophysiologically active cells.

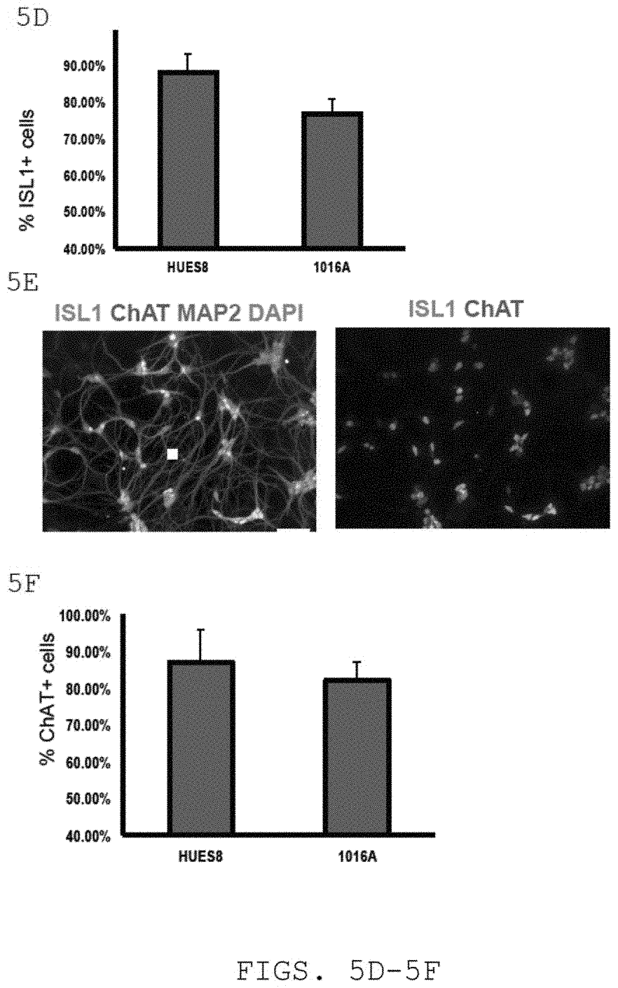

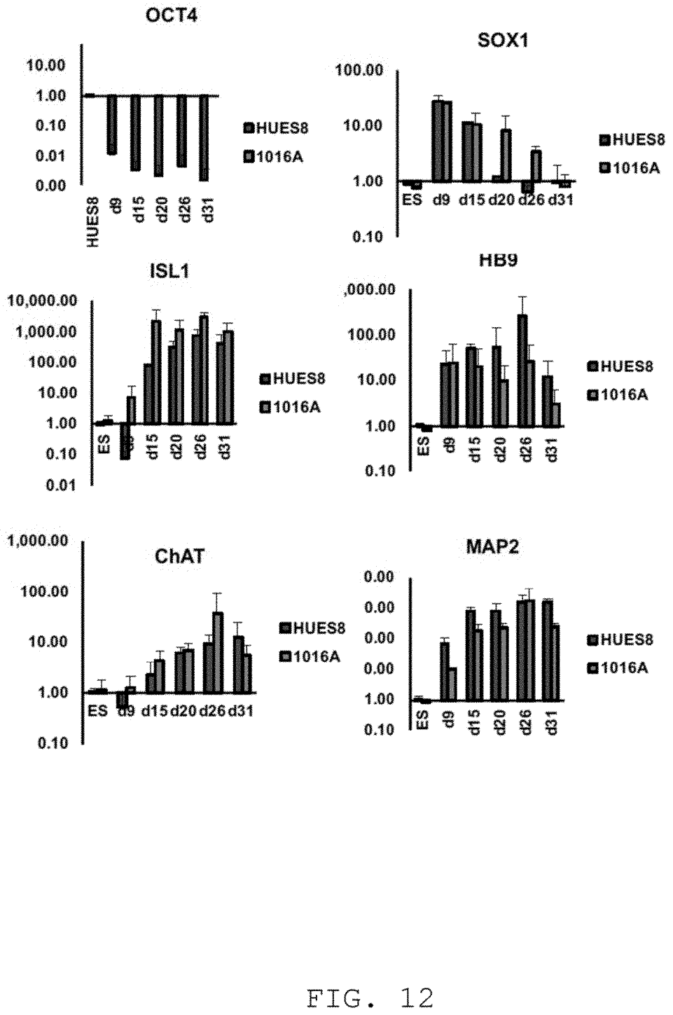

It is well known that synaptic activation through depolarization induces a transcriptional response in neurons (Ebert and Greenberg, 2013; Flavell et al., 2008; Hong et al., 2008; Lin et al., 2008). A key event in many types of neurons, including cortical neurons, is the phosphorylation of the transcription factor CREB. To examine whether neurons from dissociated cortical spheres responded to activation, we depolarized them with KCl. Immunostaining with a phospho-CREB specific antibody revealed a robust phosphorylation of CREB in these cultures (FIGS. 4A and 4B and Table 2). We then performed transcriptional analyses of KCl-stimulated cortical neurons and found increased transcription of the early-induced genes NPAS4, cFOS, and ARC by one hour after stimulation, and expression of the late-induced gene BDNF after six hours (FIG. 4C), recapitulating findings from freshly isolated rodent cortical neurons (Flavell et al., 2008; Hong et al., 2008; Lin et al., 2008). Similar results were obtained from KCl stimulation of whole spheres after 20 days of differentiation (FIG. 11).

Taken together, these data demonstrate that cortical neurons obtained from hPSCs cultured and differentiated as spheres in spinner flasks are electrophysiologically active and form neural networks in culture. Moreover, the neurons display voltage-gated Na⁺ and K⁺ currents, intracellular Ca²⁺ dynamics, and activity-induced gene expression characteristic of cortical neurons, further underlining their maturation into functional cortical neurons.

TABLE 2
Quantification of the relative proportion of neurons positive for phospho-CREB obtained from
three independent experiments. 20 days after dissociation cells were stimulated with KC1 for 30 min,
1 hr, and 6 hrs and processed for immunocytochemistry with the indicated antibodies. For each
biological replicate the total number of nuclei quantified is reported.
PCREB Cortical immunostaining

| | Tot Nuclei | O hr Kcl | Tot Nuclei | 30 min KCl | Tot Nuclei | 1 hr KCl | Tot Nuclei | 6 hr KCl |
|---|---|---|---|---|---|---|---|---|
| HUES8_1 | 22518 | 2.80% | 23713 | 38% | 21894 | 32% | 21356 | 29% |
| HUES8_2 | 27289 | 2.87% | 18662 | 45% | 43542 | 38% | 21338 | 29% |
| HUES_3 | 9335 | 3.75% | 8358 | 35% | 5191 | 34% | 3952 | 36% |
| Average | | 3.14% | | 39% | | 35% | | 31% |
| STDEV | | 0.53% | | 5% | | 3% | | 4% |

### Example 3—Motor Neuron Production from Spin Culture hPSCs

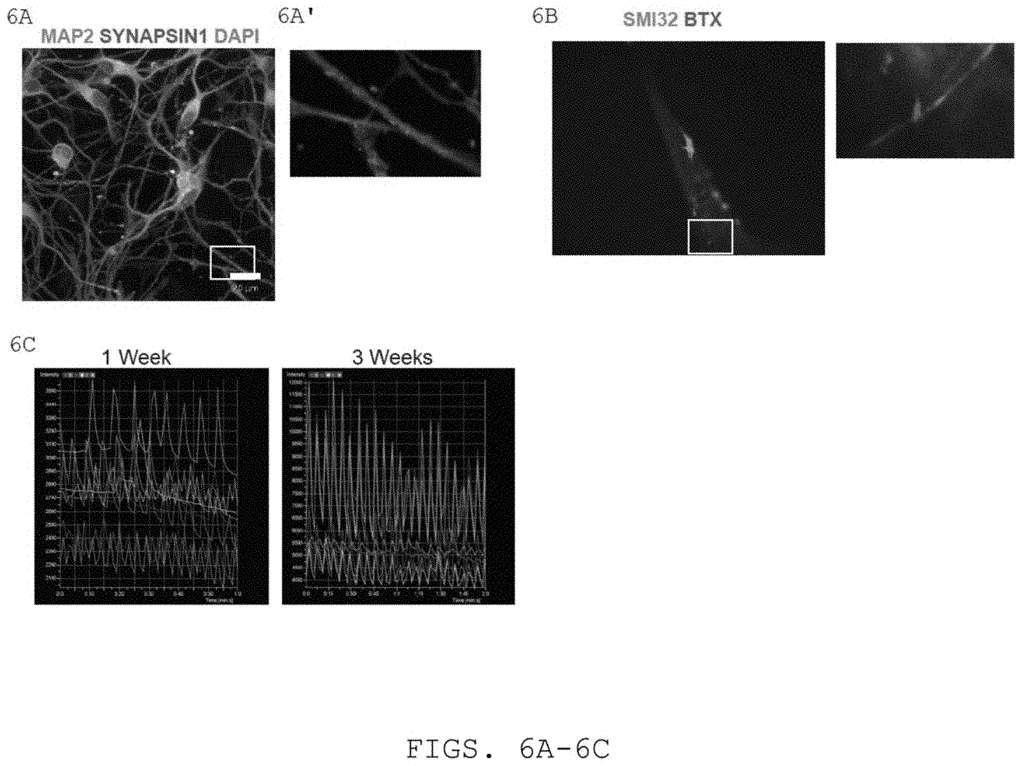

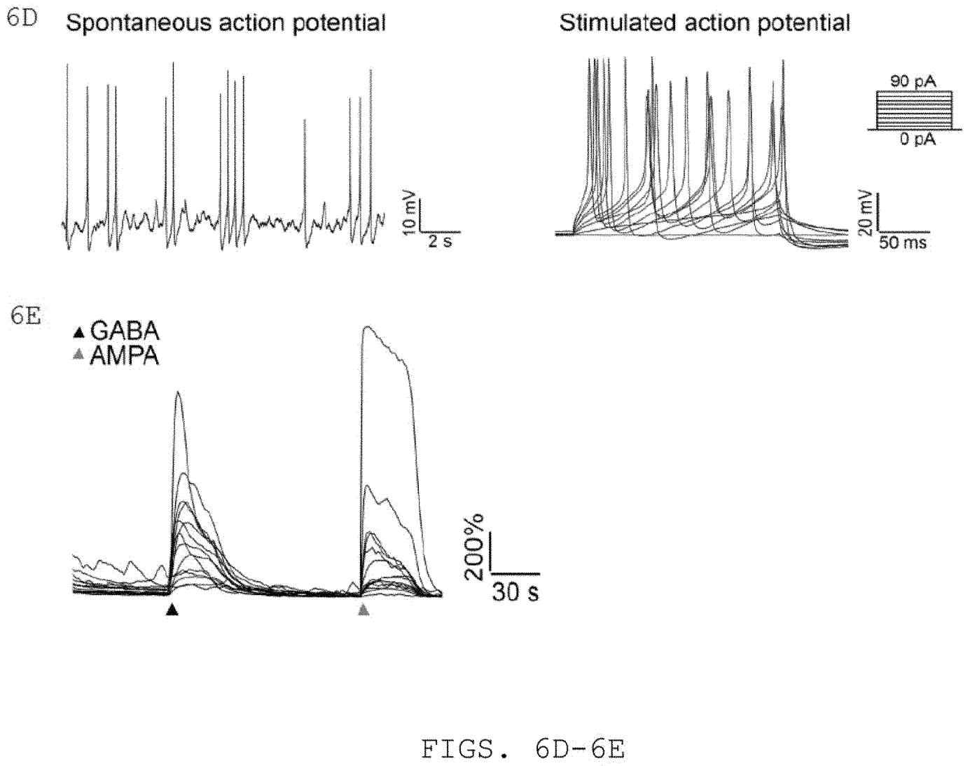

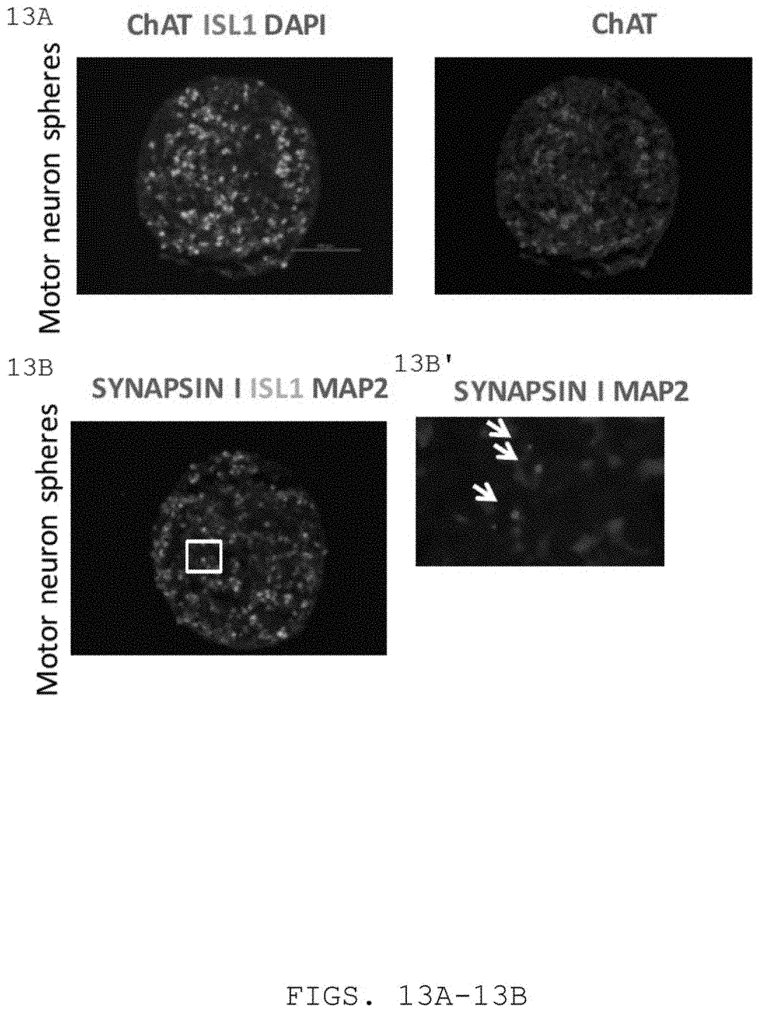

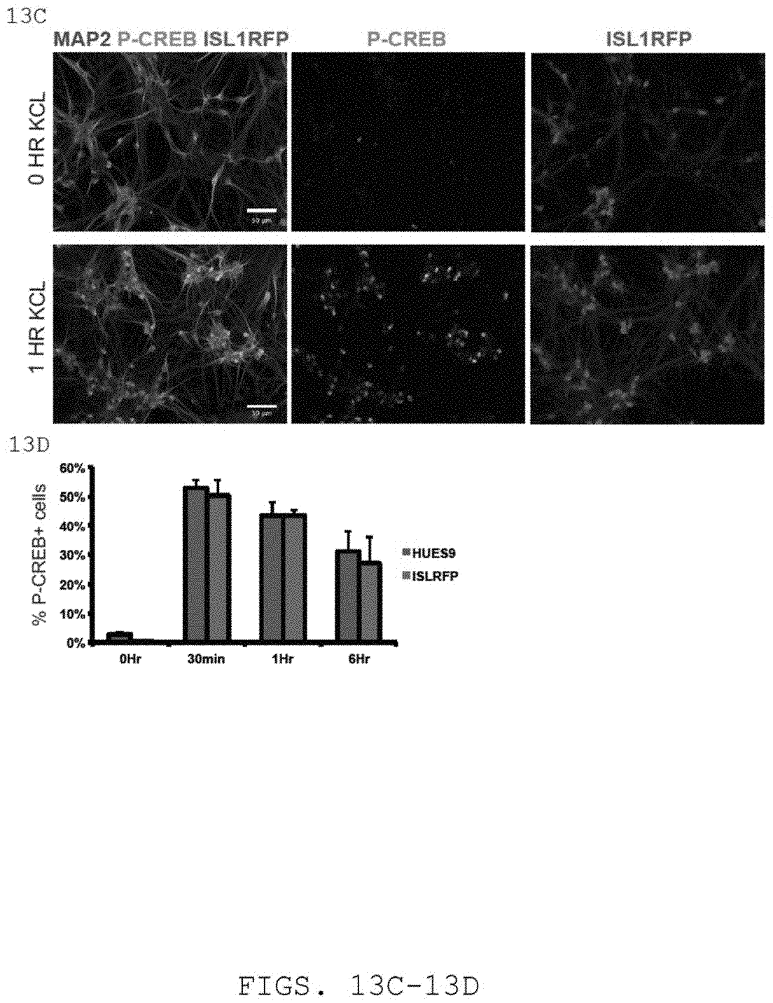

In the next set of experiments, we explored whether the spheres would respond to exogenously supplied factors by differentiating to other neuronal subpopulations. To accomplish this, we set out to modify standard motor neuron differentiation protocols (schematic in FIG. 5A). By supplying retinoic acid and sonic hedgehog (Wichterle et al., 2002), spheres differentiating toward the motor neuron fate were obtained. qRT-PCR analyses revealed a transient peak in expression of the neuronal progenitor gene SOX1 and expression of the motor neuron-specific genes ISL1 and HB9 (FIG. 12). At later time points, increasing expression of the biosynthetic enzyme ChAT, necessary for synthesis of acetylcholine, the neurotransmitter of motor neurons and neuromuscular control, and the mature pan-neuronal marker MAP2 was detected (FIG. 12). When analyzed by immunocytochemistry at day 20 of differentiation, spheres expressed ISL1, MAP2, ChAT and SYNAPSIN1 (FIG. 5B and FIGS. 13A and 13B).

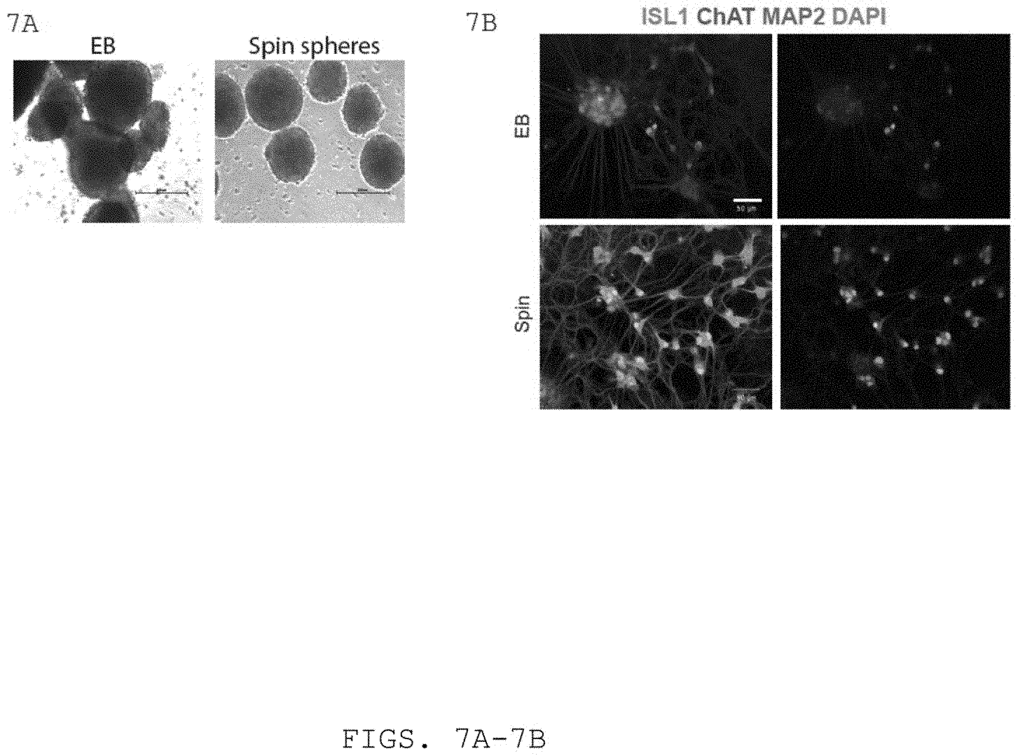

After 15 days of differentiation, motor neuron spheres were dissociated, plated and cultured under feeder-free conditions for an additional week. Immunocytochemistry revealed that up to 85% of the dissociated cells expressed ISL1, with consistent results obtained in different cell lines and low experiment-to-experiment variability (FIGS. 5C and 5D and Table 3). We also performed immunocytochemistry with an antibody specific for the enzyme ChAT (choline acetyltransferase), a biosynthetic enzyme required for cholinergic neurotransmission. The results revealed that the vast majority of motor neurons also express ChAT (FIGS. 5E and 5F and Table 3). Cells from dissociated motor neuron spheres also exhibited punctate SYNAPSIN1-immunoreactivity along MAP2-positive neurites (FIG. 6A). We also performed co-culture experiments where spin culture-derived motor neurons were cultured with human myotubes. Staining with fluorescently labeled alpha-bungarotoxin (BTX), a toxin that specifically binds to postsynaptic acetylcholine receptors, labeled regions of contact between motor neurons and muscle fibers (FIG. 6B), indicating formation of neuromuscular junctions.

TABLE 3
Quantification of the relative proportion of ISL1 and ChAT
positive cells obtained from three independent experiments in two
different hPSC lines. Cells were dissociated at day 15, plated,
cultured for 1 week, and processed for immunocytochemistry with
the indicated antibodies. For each biological replicate the total
number of nuclei quantified is reported.
Motor neurons immunostaining d 21

| d 21 | n Nuclei | % ChAT | % ISL1 |
|---|---|---|---|
| HUES8_1 | 5521.00 | 98% | 94% |
| HUES8_2 | 5473.00 | 83% | 82% |
| HUES8_3 | 2807.00 | 79% | 87% |
| Average | | 87% | 88% |
| STDEV | | 10.02% | 6.03% |
| 1016A_1 | 3795.00 | 84.00% | 72.00% |
| 1016A_2 | 4735.00 | 79.00% | 78.00% |
| 1016A_3 | 12665.00 | 82.00% | 80.00% |
| Average | | 81.67% | 76.67% |
| STDEV | | 2.52% | 4.16% |

### Example 4—Dissociated Motor Neurons Exhibit Stimulation-Induced Activity and Form Spontaneously Active Neural Networks

To further address the functionality of the motor neurons that we produced, we characterized their physiological properties. First, we asked whether these motor neurons exhibited spontaneous electrical activity in culture. As before, we monitored intracellular Ca²⁺ dynamics in cultures of dissociated motor neurons transduced with an AAV1-GCaMP6. Cells were transduced three days after dissociation and imaged every week. As early as one week after dissociation, motor neurons already exhibited significant levels of activity, with increasing fluctuations in fluorescence over time, and with groups of motor neurons exhibiting synchronized Ca²⁺ dynamics three weeks after dissociation. (FIG. 6C).

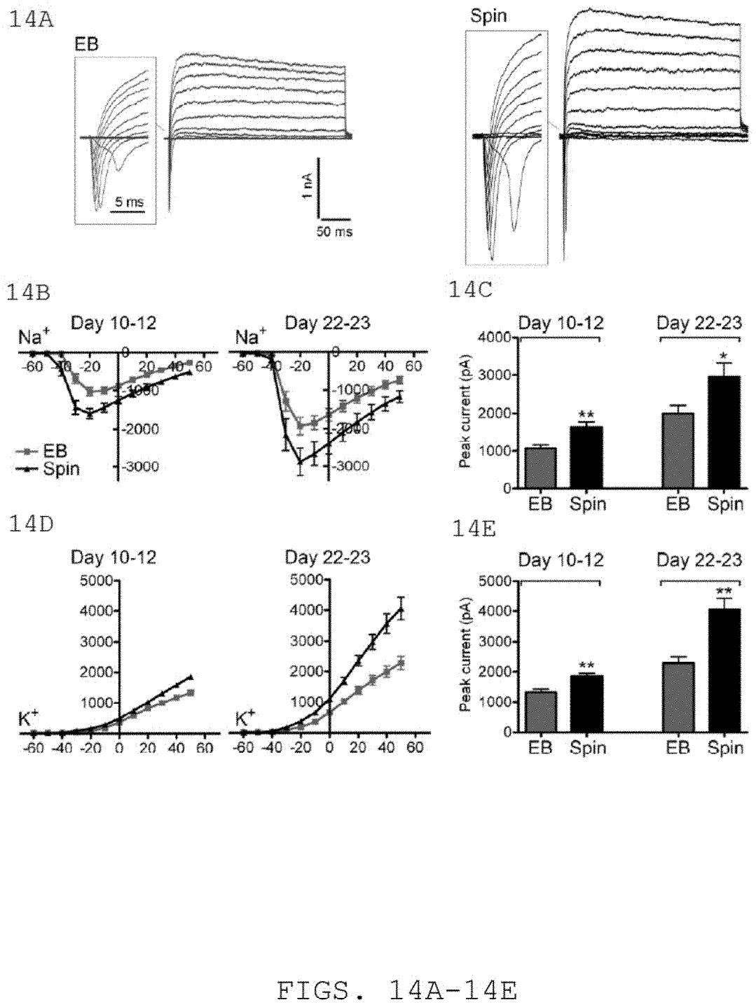

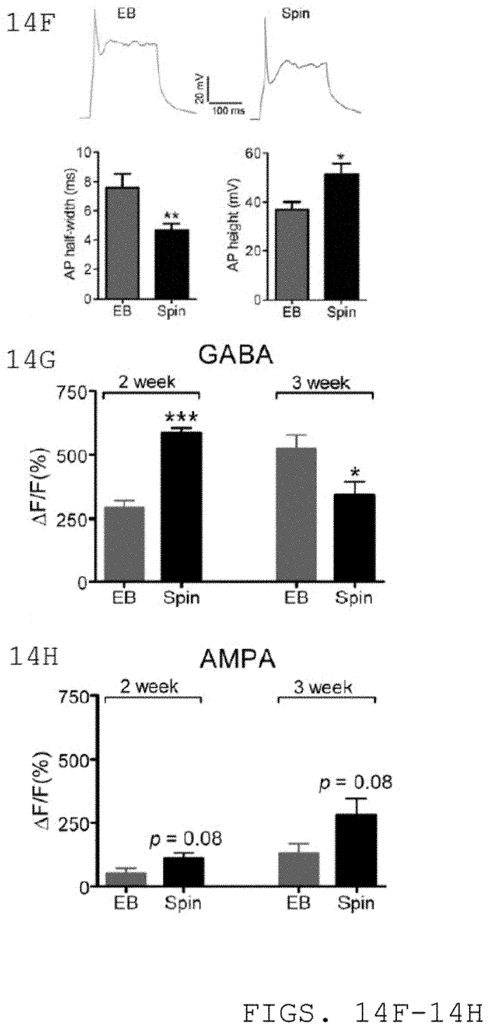

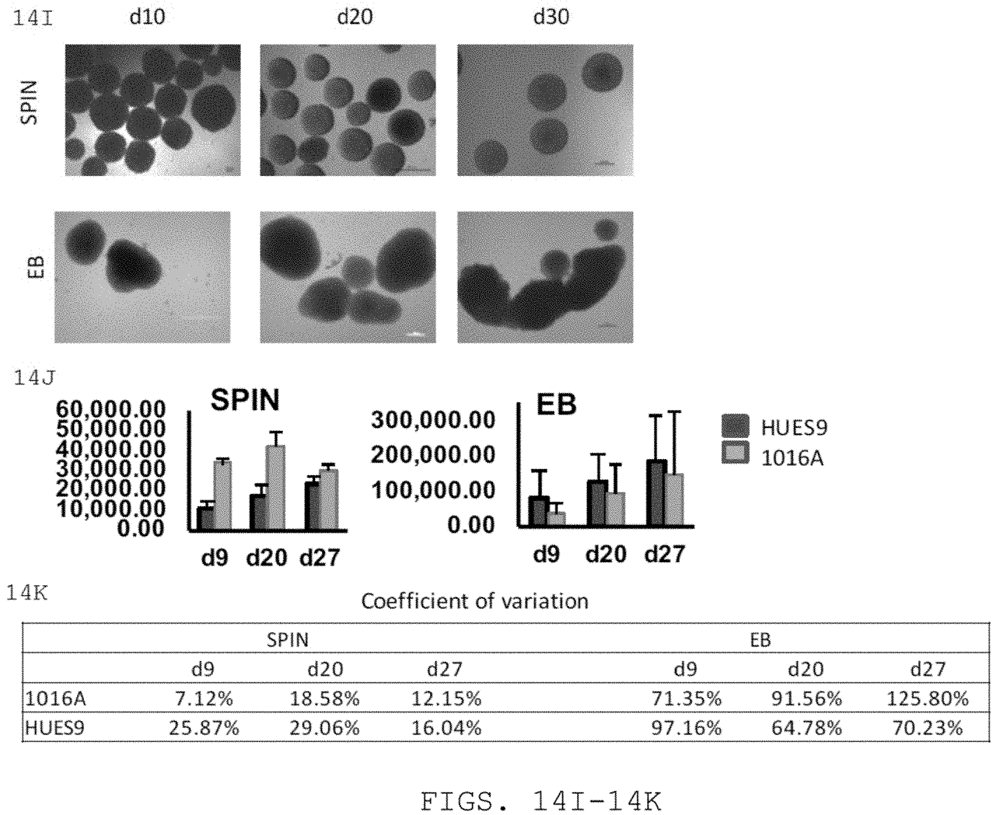

Patch-clamp recordings were performed to further understand the electrophysiological characteristics of motor neurons from dissociated spheres. Motor neurons were found to fire action potentials spontaneously and upon current injection after up to three months in culture (FIG. 6D, see also FIG. 14). Furthermore, GCaMP6-mediated monitoring of intracellular Ca²⁺ dynamics showed that motor neurons responded to GABA and AMPA, two major neurotransmitters (FIG. 6E).

Taken together, these data demonstrate that differentiating spheres respond to exogenously supplied differentiation cues. When stimulated with sonic hedgehog and retinoic acid at the proper doses and time points, differentiating cells within the spheres adopt the motor neuron fate and can be dissociated and replated without glial feeder layers. Importantly, motor neurons cultured for extended periods of time showed spontaneous firing of repetitive action potentials, suggesting functional maturation after long-term culture. Furthermore, immunocytochemistry revealed that motor neurons responded to KCl depolarization by phosphorylation of CREB (FIGS. 13C and 13D), documenting proper intracellular response to membrane depolarization.

Thus, the sphere differentiation protocol was successfully modified to allow the generation of billions of motor neurons from one single differentiation experiment. The yield of motor neurons was very efficient, up to 85%, with little variation between individual experiments. Moreover, the obtained motor neurons expressed ChAT, and co-cultures with skeletal muscle fibers resulted in BTX-labeling of regions of contact between motor neurons and muscle fibers, suggesting formation of neuromuscular junctions. The motor neurons fired action potentials upon current injection and exhibited spontaneous action potentials.

### Example 5—Increased Survival and Function of Neurons Obtained Through the Spin Culture Protocol Compared to EB-Derived Motor Neurons

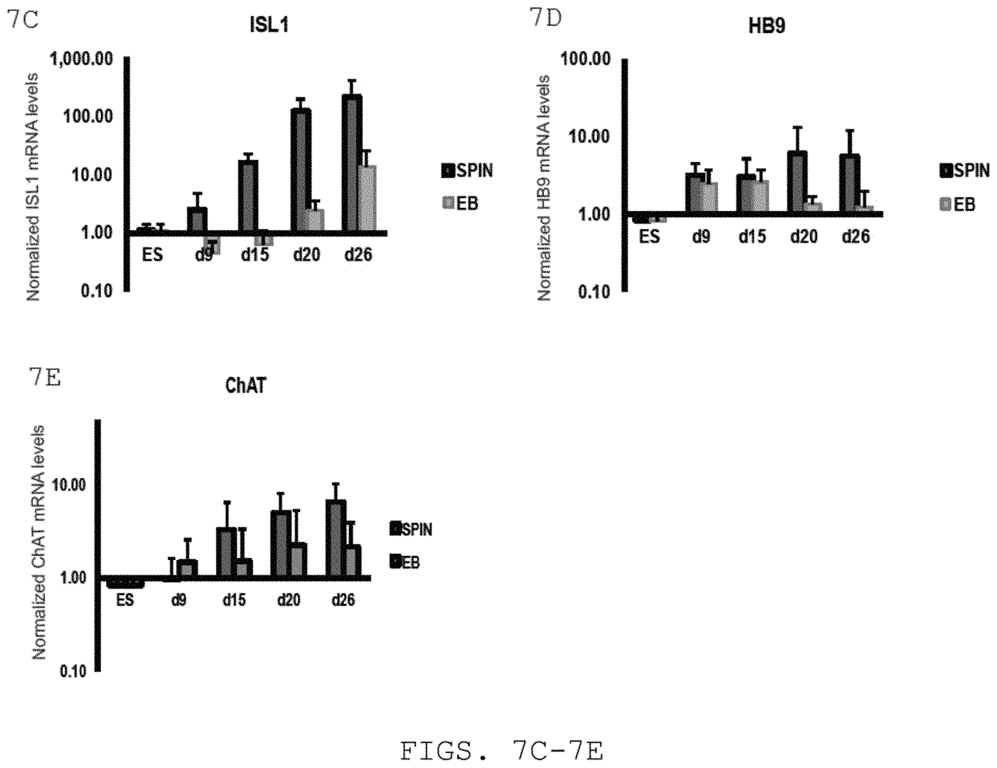

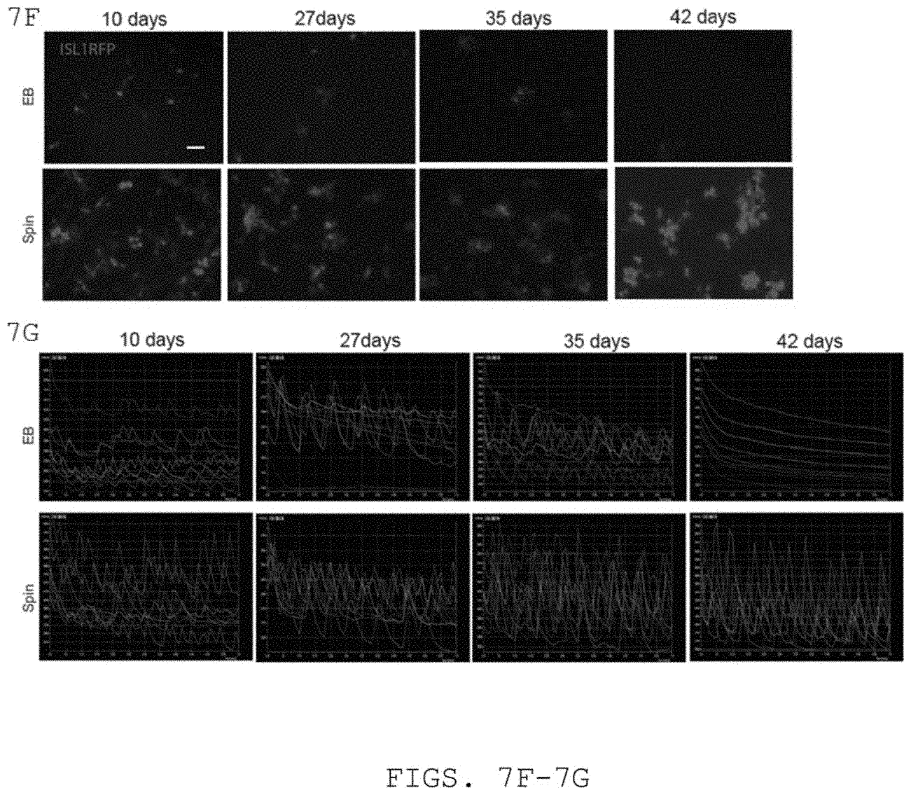

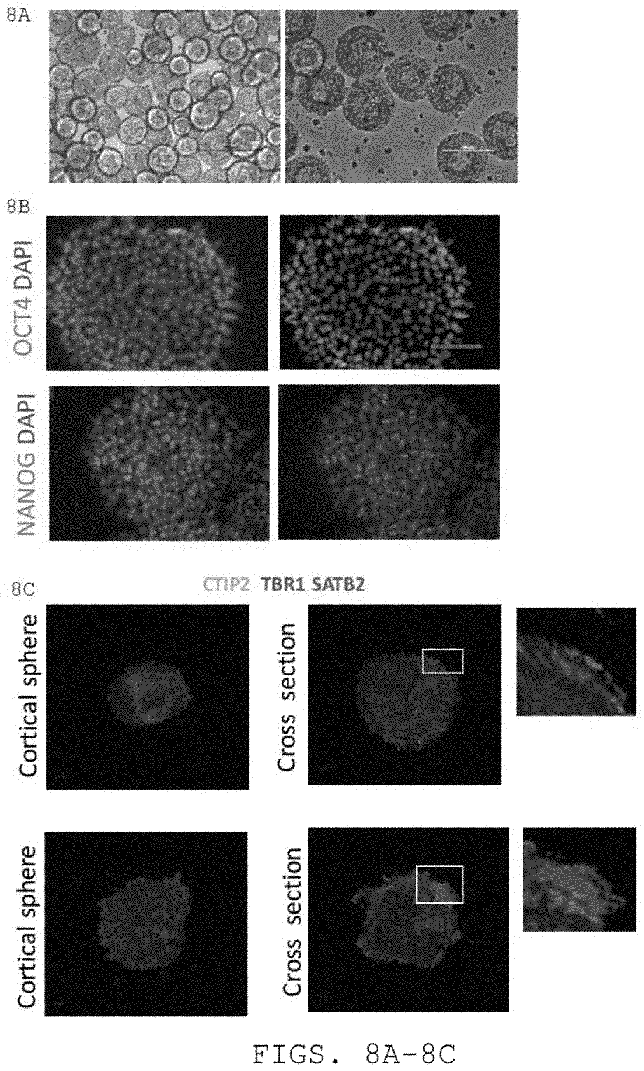

Throughout the course of the experiments presented, we noted a marked increase in survival of spin culture-derived neurons compared to EB-derived neurons in all assays requiring manipulations such as dissociation, FACS, and viral transduction. In an attempt to address this in a quantifiable manner, we generated motor neurons in parallel using two methods (but with exactly the same medium composition): spin culture differentiation and through EB formation in low attachment tissue culture plates. Motor neuron spheres in spin culture were uniform and homogenous, whereas motor neuron differentiation using EBs led to formation of cell clumps exhibiting marked heterogeneity with respect to size, shape, and general appearance (FIG. 6A and FIGS. 14I-14K). Expression analyses by qRT-PCR of cells undergoing differentiation at different time points revealed that motor neuron spheres exhibited higher levels of expression of ISL1, HB9, and ChAT compared to conventional EB-derived motor neurons (FIGS. 7C-7E). When spheres were dissociated and motor neurons plated, immunocytochemistry showed that spin culture-derived motor neurons co-expressed ISL1, MAP2, and ChAT to a higher degree than EB-derived motor neurons. (FIG. 7B).

Next, we performed a set of experiments where we monitored the appearance and functionality of cultures of motor neurons derived from spin culture spheres or using EB methodology. Irrespective of plating conditions, motor neurons derived from spin culture spheres survived for a longer period of time in culture. The difference was particularly striking on coated tissue culture plates (FIG. 7F), where motor neurons from dissociated spin culture spheres could be maintained in culture, without glial feeder layers, for several months, which in our hands proved to be impossible using motor neurons obtained through EB formation. In experiments where motor neurons were plated and transduced with genetically encoded Ca²⁺ sensors, motor neurons derived from spin culture spheres exhibited higher amplitude and frequency in levels of fluorescence compared to EB-derived motor neurons (FIG. 7G). With extended time in culture, the amplitude and frequency of fluctuations in Ca²⁺ increased in the spin culture-derived motor neurons, whereas EB-derived motor neurons exhibited a loss in Ca²⁺ fluctuations (FIG. 7G). These results are consistent with the notion that motor neurons derived from spin culture spheres survive better and maintain their functionality during extended periods of culture.

To formally address the maturation of the different motor neuron populations, we performed comparisons using whole cell patch recordings (FIG. 14). Our recordings of action potentials and voltage-gated Na/K currents showed that spin culture-derived motor neurons were able to fire faster action potential and larger Na/K currents than EB-derived motor neurons (FIGS. 14A-14F). In addition, calcium imaging experiment suggest that spin culture-derived motor neurons express functional GABA receptors and begin to undergo the transition from GABA excitation to inhibition earlier than EB-derived motor neurons (FIGS. 14G-14H). These data indicate that spin culture-derived motor neurons appear to mature faster than EB-derived motor neurons in regards to their electrophysiological characteristics, but additional experiments in more hPSC lines are required to address whether this is a general phenomenon.

### Discussion

In the last three years, several groups have shown that neural organoids, 3D spheres of ex vivo-derived nervous tissue with features of mammalian CNS, can be generated (Bershteyn and Kriegstein, 2013; Lancaster et al., 2013; Sasai, 2013). This innovative approach has opened up the possibility of studying basic mechanisms of the developmental biology of the human nervous system and neurodevelopmental disorders. However. it is important to note that there are limitations also with this model system. Neural organoids are produced from hPSC through formation of neural progenitors that subsequently are aggregated individually in Matrigel into spheres that are cultured in spinner flasks. The resulting organoids are highly complex structures containing many different types of neurons organized into areas exhibiting features of distinct regions of the mammalian CNS, including the cerebral cortex. However, neural differentiation in organoids is quite variable both with respect to the exact type of neuronal subtype and structure produced, as well as the efficiency of the differentiation process.

In this paper, we present a new method based on 3D growth of cells from hPSC to differentiated neuron. Importantly, this protocol does not rely on isolation and manipulation of an intermediate cell population, such as sphere formation of neural progenitors in Matrigel required for neural organoid formation. In contrast, we culture cells as spheres already in the pluripotent state and only subject the spheres to differentiation cues by changing the composition of the culture medium. For both cortical neurons and motor neurons produced with this method, we were able to document not only expression of appropriate marker genes, but also several hallmarks of mature, functional neurons, including synapse formation, expression of functional receptors for neurotransmitters, spontaneous electrical activity, and appropriate expression of activity-induced genes.

This system has several advantages. First, it is simple to obtain a high absolute number of cells in the range of billions even in a single flask. This is of importance for downstream applications such as high throughput screening, where cell numbers are a limiting factor. For example, our laboratory routinely performs chemical screens on hPSC-derived neurons in 384-well plates, where 15,000 neurons are plated in each well (Yang et al., 2013). One single differentiation of hPSC spheres in spinner flasks as described in this manuscript would be sufficient for approximately 350×384-well plates, thereby greatly reducing the time and effort required to produce the cells necessary to conduct such experiments. Second, spheres cultured in the spinner flasks appear uniform in size and shape during the differentiation, reducing experiment-to-experiment variation. Third, spin culture differentiation results in increased relative numbers of cortical neurons and motor neurons. Fourth, after dissociation of the differentiated spheres, we were able to culture both types of neurons without the use of feeder layer for extended periods of time, up to 4 months. This is in stark contrast to neurons obtained from EBs, where cells start to detach from the plate. These features may facilitate the study of events that have been challenging to reproduce in cell culture, such as those associated with late onset disease.

In this report we have focused on generating cortical and motor neurons, but we believe it is likely that future adaptions of the sphere protocol to generate other distinct neuronal subtypes will prove to be advantageous compared to other methods in a similar manner. In addition, adapting imaging and physiological assays to cells maintained in 3D should be possible in the near future so that functional connections among populations of neurons can be maintained and analyzed.

### Experimental Procedures

### Media

mTeSR (Stem Cell Technologies); KSR medium: [15% KSR (Invitrogen), KO DMEM (Invitrogen), L-Glutamine (Gibco), 100×NEAA (Gibco), 100× Penicillin-Streptomycin (Gibco), and 1000× beta mercaptoethanol (Gibco)]; Neural induction medium (NIM): [DMEM/F12 (Invitrogen), 100×N2 supplement (Gibco), 50×B27 supplement (Life Technologies) (for cortical differentiation we substituted regular B27 for B27 supplement without vitamin A (Life Technologies)), 100× Glutamax (Gibco), 100×NEAA (Gibco), 100× Penicillin-Streptomycin (Gibco), 125× Glucose (J.T.Baker), for only MN 0.2 mM Ascorbic Acid (Sigma)]; Neurobasal (NB) medium: [Neurobasal (Invitrogen), 100×N2 supplement (Gibco), 50×B27 supplement (Life Technologies) (for cortical differentiation we substituted regular B27 for B27 supplement without vitamin A (Life Technologies)), 100× Glutamax (Gibco), 100×NEAA (Gibco), 100× Penicillin-Streptomycin (Gibco). For motor neurons, the media was supplemented with 125× Glucose (J.T. Baker), and 0.2 mM Ascorbic Acid (Sigma)].

hPSC Adaption and Maintenance in Spinner Flasks

hPSCs were cultured in 500 mL disposable spinner flasks (Corning, VWR) on a 9-position stir plate (Dura-Mag) at a rotation rate of 70 rpm, in a 37° C. incubator with 5% CO₂. Prior to adaption to spinner flask culture, hPSCs were expanded in 10 cm dishes (Corning) and dissociated to single cells with Accutase (Stemcell Technologies), 5 min incubation at 37° C., when they were approaching confluency. 180 million individual pluripotent stem cells were seeded into a spinner flask in 180-200 mL of mTeSR medium (Stemcell Technologies) supplemented with ROCK inhibitor 5 μM Y-27632 (Stemcell Technologies). Spheres formed naturally and after 2 days media was changed. The cells were maintained as undifferentiated pluripotent spheres in spin culture, with medium changes every day. Every third day, when the spheres were approximately 50-100 μm in diameter, cells were passaged by incubating at 37° C. for 6 minutes in a 1:1 mixture of Accutase and PBS, and by mechanically dissociating the cells. Cells were filtered and re-seeded into the flask at the same initial density as described above. Medium was changed by taking the flasks off of the stir plate, allowing the cells to settle to the bottom of the flask, and the medium to be changed. At day 1 of differentiation, medium was changed to mTeSR with the activin/TGF-β inhibitor SB431542 (R&D System) (10 μM) and the BMP inhibitor LDN193189 (Stemgent) (100 nM) (hereafter referred to as “dual SMAD inhibition”). Subsequent steps of the differentiation protocol (as described below) use the same method for changing media.

### Differentiation Conditions

Cortical Differentiation:

from day 2 to day 10, spheres were gradually adapted to NIM through a dilution series of KSR and NIM, with dual SMAD inhibition maintained until day 7. Media was changed as follows: d2, d3, d4: 100% KSR media with the Wnt signaling inhibitor XAV939 (2 μM) (Stemgent); d5: 75% KSR media, 25% NIM; d7: 50% KSR, 50% NIM; d9: 25% KSR, 75% NIM. From day 11 until day 21, cultures were maintained in 100% NIM (Maroof et al., 2013). From day 21 onwards, cultures were maintained in NB medium with brain-derived neurotrophic factor (BDNF) and glial cell-derived neurotrophic factor (GDNF) (both from R&D, 10 ng/ml).

Motor Neuron Differentiation:

from day 2 to day 10, spheres were gradually adapted to NIM through a dilution series of KSR and NIM, with dual SMAD inhibition maintained until day 6. Media changed as follows: d2: 100% KSR media; d3: 100% KSR media+retinoic acid (RA) (Sigma, 1 μM); d5: 75% KSR media, 25% NIM+RA, BDNF (R&D, 10 ng/ml); d6: 50% KSR, 50% NIM+RA, Smoothened agonist (SAG) (Curis, 1 μM), BDNF; d8: 25% KSR, 75% NIM+RA, SAG, BDNF. From day 10 until day 15, cultures were maintained in 100% NIM+RA, SAG, BDNF, DAPT (R&D System, 2.5 μM). From day 15 onwards, cultures were maintained NB medium+BDNF, GDNF, and ciliary neurotrophic factor (CNTF) (R&D System 10 ng/ml).

During the differentiation, spheres were collected at different time points and RNA was extracted for qRT-PCR analysis. For cortical differentiation, spheres were dissociated with Accutase and plated onto Matrigel-coated plates at day 13 and analyzed the following day for expression of cortical progenitor markers by immunofluorescence. Spheres were also dissociated at day 40 with 0.05% Trypsin (Gibco) and plated onto poly-D-lysine/Laminin (EMD Millipore/Life technologies)-coated plates. After 28 and 43 days of additional culture, cells were analyzed as described below. For motor neurons differentiation, spheres were dissociated with 20 units of papain per ml in EBSS supplemented with 1 mM L-cysteine, 0.5 mM EDTA (Worthington), and DNAse. 60,000 cells/well plate were plated onto 96-well poly-D-lysine/Laminin-coated plates at day 15 (See a detailed protocol for dissociation in the additional experiment procedures in the supplemental information accompanying this manuscript). Before dissociation, both cortical and motor neuron spheres were approximately 200 μm in diameter. Neurons were analyzed after one additional week in culture post dissociation.

### Statistics

A two-tailed Student's t test was used to calculate statistical significance.

### Additional Experimental Procedures

### Cells and Cell Culture

All cell cultures were maintained at 37° C. with 5% CO₂. Before being adapted to spin culture conditions, human ESCs/iPSCs were cultured and expanded on Matrigel (Corning)-coated plates in mTeSR medium (Stem Cell Technologies) with FGF Stem Beads (Stem Culture, 4 μL/ml). Media was changed every other day. Cells were passaged with Accutase (Stemcell Technologies) for 5 min at 37° C. The following lines were used: HUES8, HUES9, BJ (siPS), for cortical differentiation; HUES8, HUES9, 1016A (all from Harvard University) and H9ISLRFP (Bu et al., 2009) for motor neuron differentiation. All experiments with the human ES cell lines were reviewed and approved by the Harvard Embryonic Stem Cell Oversight Committee. The use of the wild-type iPS cell lines by the Rubin lab was reviewed by the Harvard Committee on the Use of Human Subjects (the Harvard IRB) and determined to not constitute human subjects research.

### Cortical Neuron Dissociation for Spin Culture Spheres

Spheres were collected at day 40 of differentiation, washed with PBS and incubated with Trypsin 0.05% (Gibco) and DNAse (Worthington, 1000 units) for 10 min at 37° C. under gentle mutation. Cells were washed with PBS, pelleted, and re-suspended in NB media and gently dissociated mechanically by gentle pipetting. Cells were washed several times, counted, filtered, resuspended in NB media with BDNF and plated in plates coated with laminin (0.025 mg/mL) and poly-D-lysine (0.5 mg/mL) (EMD Millipore/Life technologies) as follows: for 96-wells plates, 50,000 cells/well; 24-well plates, 200,000 cells/well; 6-well plates, 1.4×10⁶ cells/well.

### Human Motor Neurons Dissociation for EBs and Spin Culture Spheres

Spin culture spheres and EBs were collected at day 15 of differentiation and dissociated with papain, DNAse, and ovomucoid (Worthington) prepared as recommended by the manufacturer. Spin culture spheres and EBs were washed once with PBS and incubated with papain and DNAse (100 units and 1000 units, respectively, per 0.5 mL pellet of spheres or 2×10 cm dishes of EBs) for 10 min at 37° C. under gentle mutation. Ovomucoid inhibitor was added at a final concentration of 10%, and the cells were pelleted and gently resuspended in 2 mL of EBSS supplemented with DNAse (1000 units). Aggregates were allowed to separate and settle for a few minutes while single cells were collected. Additional EBSS/DNAse was added to the remaining aggregates and the process was repeated six to seven times until no more aggregates were seen. Cells were washed twice in NB media, counted, resuspended in NB media supplemented with BDNF, CTNF and GDNF, filtered, and plated in plates coated with laminin (0.025 mg/mL) and poly-D-lysine (0.5 mg/mL) as follows: for 96-wells plates, 60,000 cells/well; 24-well plates, 300,000 cells/well; 6-well plates, 1.6×10⁶ cells/well.

### Immunohistochemistry

Cell cultures were fixed in 4% paraformaldehyde (PFA) in PBS at room temperature (RT) for 20 min, then blocked for 20 min in 5% horse serum (HS) (Gibco) and 0.1% Triton X-100 (Sigma) in PBS. Cells were incubated with primary antibodies overnight at 4° C. Secondary antibodies were applied for 1 hr at room temperature. Whole spheres were cleared using the SeeDB protocol as follows: samples were fixed in 4% PFA and blocked for 24-48 hrs in 0.1% Triton X-100 and 5% HS at 4° C. Samples were then incubated with primary antibodies for 2-3 days at 4° C. in either blocking solution or 10% HS. Samples were washed with PBS 3 times and incubated with secondary antibodies for 3 days in either blocking solution or 10% HS. Following washing with PBS, samples were serially incubated in 20%, 40%, 60%, and 80% (wt/vol) fructose for 6-12 hrs at 25° C. Samples were immersed in 100% (wt/vol) fructose for 24 hrs, followed by SeeDB (115%, wt/vol) for at least 24 hrs at 25° C. All solutions were prepared with H₂O and contained 0.5% α-thioglycerol. Samples were stored in SeeDB at 4° C.

The primary antibodies used in this study were raised against PAX6 (Covance), OTX1-2, Ki67, TBR1, SATB2, CTIP2, and ISL1 (Abcam), GLUR1, and ChAT (Millipore), SYNAPSINI (Synaptic System), TUJ1 (Covance), and MAP2 (Lifespan Biosciences). Secondary antibodies were AlexaFluor488, -546, -555, and -647-conjugated antibodies raised against the appropriate species (Invitrogen). Immunocytochemical images were acquired on the Opera High-Content Screening System (PerkinElmer). Immunohistochemical images and Ca²⁺ imaging was acquired on the Nikon Eclipse TE2000-S microscope equipped with a heated stage and a humidified chamber with atmospheric control. 3D LSFM was performed on the ZEISS Lightsheet.Z1 Microscope Quantitative image analysis of neuronal cultures was done using PerkinElmer Columbus Image Data Storage and Analysis System v2.3.3.

qRT-PCR

Total RNA was isolated using RNeasy mini kit (QIAGEN) and subsequent cDNA synthesis with iSCRIPT (Bio-Rad). qRT-PCR was then performed using SYBER green (Bio-Rad) on a QuantStudio™ 12 K Flex Real-Time PCR System (Life Technologies). Quantitative levels for all genes were normalized to endogenous GAPDH. Expression levels of individual genes were normalized to the levels in undifferentiated human ESC/iPSC of each line. For KCL experiments, expression levels were normalized to the levels in untreated cells.

### KCl Experiments

KCl experiments were performed on spheres at day 20 of differentiation and on dissociated neurons 10 days after dissociation. Neurons were silenced overnight with TTX (Natchannel-blocker) (EMD Bioscience, 1 μM) and AP-V (NMDAR antagonist) (Sigma Aldrich, 100 μM), two compounds that together inhibit any spontaneous activity in cultured neurons. The next day KCl (60 mM) was added to the silenced neurons for 30 minutes, 1 hour, or 6 hours to depolarize the cell membrane by opening voltage-gated channels. qRT-PCR analysis of cDNA collected after the designated time points was performed to see whether previously documented activity-induced genes (BDNF, NAPS4, cFOS, and ARC) were expressed. Phospho-CREB was detected by immunostaining as described.

### Ca2+ Imaging

Differentiated neurons were transduced with AAV1.Syn.GCaMP6s.WPRE.SV40 (Penn Vector Core, AV-1-PV2824) 3 days after plating to express the genetically encoded calcium sensor GCaMP6s under the control of human SYNAPSIN1 promoter. Every 10 days post-infection, cells were imaged and analyzed with a Nikon Eclipse TE2000 microscope. Images were obtained by time lapse microscopy, with one image captured every second for one minute. Intensity of fluorescence was plotted as a function over time using Nikon Element software.

### Electrophysiology

hPSC-derived neurons were dissociated and seeded on coverslips in 24-well plates at a density of 300,000 cells per well. Coverslips were coated with poly-D-lysine and laminin. Whole-cell patch clamp recordings were performed using a Multiclamp 700B amplifier and a Digidata 1550 Digitizer (Molecular Devices). Data were collected using pClamp 10 software (Molecular Devices, Sunnyvale, Calif.), sampled at 10 kHz, and filtered at 1 kHz. During recording, cells were continually perfused with bath solution (in mM) at room temperature: 128 NaCl, 30 glucose, 25 HEPES, 5 KCl, 2 CaCl₂, and 1 MgCl₂ (adjusted to pH 7.3 with NaOH). Patch pipettes were pulled from borosilicate glass using a P-1000 Micropipette Puller (Sutter Instrument) with resistance of 4-8 MΩ when filled with pipette solution containing (in mM): 147 KCl, 5 Na_{z}-phosphocreatine, 2 EGTA, 10 HEPES, and 2 MgATP, 0.3 Na₂GTP (adjusted to pH 7.3 with KOH). The series resistance was 10-25 MΩ. To record voltage-dependent sodium and potassium currents, the membrane potential was depolarized (300 ms duration) from −60 mV to 50 mV in 10 mV increments with a holding potential of −70 mV. Leak current was subtracted using an online P/8 protocol. Spontaneous action potentials were recorded without current injection. To evoke action potentials, the membrane potential was held at about −60 mV, and depolarizing current pulses were injected from 0 to 90 pA in 10 pA increments. The criterion for identifying action potential is a rapid increase of membrane potential over 0 mV. Quantifications of sodium/potassium current and action potential properties were performed by Clampfit 10.5 (Molecular Devices).

### GABA Switchover Experiments

For Ca²⁺ imaging during the GABA switchover experiment cells were transduced with AAV1.Syn.GCaMP6s.WPRE.SV40 4 days after plating. During recording, coverslips were transferred to a perfusion chamber mounted on a Nikon Eclipse TE2000 microscope. Time-lapse recordings were performed at RT at a rate of 1 frame per second. GABA and AMPA were prepared and applied for 10 seconds in the same bath solution as used for electrophysiology. Mean fluorescent intensity of GCaMP6 fluorescence was measured in soma by ImageJ (U.S. National Institutes of Health, available at the world wide web at subdomain //imagej.nih.gov/ij/) and plotted by Igor. The amplitude was normalized to baseline fluorescent level (mean of 5 frames before drug application) for each cell. Background fluorescent level was determined in a cell-free area for each recording and subtracted.

### NMJ Formation

Human skeletal myoblasts (Gibco) were cultured on laminin (0.025 mg/mL) coated plates and differentiated in DMEM+2% horse serum for 5 days. Dissociated HUES8-derived motor neurons were subsequently plated (2×10⁶/mL) on differentiated human myoblasts and cultured for an additional 10 days in NB medium supplemented with GDNF, BDNF, and CNTF. Cells were fixed in 4% PFA and stained using Alexa555-conjugated alpha bungarotoxin (Invitrogen) to localize the NMJ and using an antibody specific for SMI32 to identify neuronal projections.

### EB and Spin Culture Sphere Size Quantification

Brightfield images of EBs and spin culture spheres were converted to 8-bit black and white images and analyzed in ImageJ. Briefly, the threshold was adjusted using the Image→Adjust→Threshold command, and thereafter the surface area was determined using the Analyze→Analyze Particles command. Coefficient of variance was calculated for each hPSC line and each time point by dividing the obtained standard deviation and average.

### REFERENCES

Amoroso, M. W., Croft, G. F., Williams, D. J., O'Keeffe, S., Carrasco, M. A., Davis, A. R., Roybon, L., Oakley, D. H., Maniatis, T., Henderson, C. E., et al (2013). Accelerated high-yield generation of limb-innervating motor neurons from human stem cells. The Journal of neuroscience: the official journal of the Society for Neuroscience 33, 574-586.
Ben-Ari, Y. (2002). Excitatory actions of gaba during development: the nature of the nurture. Nature reviews Neuroscience 3, 728-739.
Bershteyn, M., and Kriegstein, A. R. (2013). Cerebral organoids in a dish: progress and prospects. Cell 155, 19-20.
Boissart, C., Poulet, A., Georges, P., Darville, H., Julita, E., Delorme, R., Bourgeron, T., Peschanski, M., and Benchoua, A. (2013). Differentiation from human pluripotent stem cells of cortical neurons of the superficial layers amenable to psychiatric disease modeling and high-throughput drug screening. Translational psychiatry 3, e294.
Boulting, G. L., Kiskinis, E., Croft, G. F., Amoroso, M. W., Oakley, D. H., and Wainger, B. J. (2011). A functionally characterized test set of human induced pluripotent stem cells. Nature biotechnology 29, 279-286.
Bu, L., Jiang, X., Martin-Puig, S., Caron, L., Zhu, S., Shao, Y., Roberts, D. J., Huang, P. L., Domian, I. J., and Chien, K. R. (2009). Human ISL1 heart progenitors generate diverse multipotent cardiovascular cell lineages. Nature 460, 113-117.
Chen, T. W., Wardill, T. J., Sun, Y., Pulver, S. R., Renninger, S. L., Baohan, A., Schreiter, E. R., Kerr, R. A., Orger, M. B., Jayaraman, V., et al. (2013). Ultrasensitive fluorescent proteins for imaging neuronal activity. Nature 499, 295-300.
Di Giorgio, F. P., Boulting, G. L., Bobrowicz, S., and Eggan, K. C. (2008). Human embryonic stem cell-derived motor neurons are sensitive to the toxic effect of glial cells carrying an ALS-causing mutation. Cell stem cell 3, 637-648.
Dolmetsch, R., and Geschwind, D. H. (2011). The human brain in a dish: the promise of iPSC-derived neurons. Cell 145, 831-834.
Ebert, D. H., and Greenberg, M. E. (2013). Activity-dependent neuronal signalling and autism spectrum disorder. Nature 493, 327-337.
Eiraku, M., and Sasai, Y. (2012). Mouse embryonic stem cell culture for generation of three-dimensional retinal and cortical tissues. Nature protocols 7, 69-79.
Eiraku, M., Watanabe, K., Matsuo-Takasaki, M., Kawada, M., Yonemura, S., Matsumura, M., Wataya, T., Nishiyama, A., Muguruma, K., and Sasai, Y. (2008). Self-organized formation of polarized cortical tissues from ESCs and its active manipulation by extrinsic signals. Cell stem cell 3, 519-532.
Espuny-Camacho, I., Michelsen, K. A., Gall, D., Linaro, D., Hasche, A., Bonnefont, J., Bali, C., Orduz, D., Bilheu, A., Herpoel, A., et al. (2013). Pyramidal neurons derived from human pluripotent stem cells integrate efficiently into mouse brain circuits in vivo. Neuron 77, 440-456.
Flavell, S. W., Kim, T. K., Gray, J. M., Harmin, D. A., Hemberg, M., Hong, E. J., Markenscoff-Papadimitriou, E., Bear, D. M., and Greenberg, M. E. (2008). Genome-wide analysis of MEF2 transcriptional program reveals synaptic target genes and neuronal activity-dependent polyadenylation site selection. Neuron 60, 1022-1038.
Fox, I. J., Daley, G. Q., Goldman, S. A., Huard, J., Kamp, T. J., and Trucco, M. (2014). Stem cell therapy. Use of differentiated pluripotent stem cells as replacement therapy for treating disease. Science 345, 1247391.
Gao, B. X., and Ziskind-Conhaim, L. (1998). Development of ionic currents underlying changes in action potential waveforms in rat spinal motoneurons. Journal of neurophysiology 80, 3047-3061.
Han, S. S., Williams, L. A., and Eggan, K. C. (2011). Constructing and deconstructing stem cell models of neurological disease. Neuron 70, 626-644.
Hester, M. E., Murtha, M. J., Song, S., Rao, M., Miranda, C. J., Meyer, K., Tian, J., Boulting, G., Schaffer, D. V., Zhu, M. X., et al. (2011). Rapid and efficient generation of functional motor neurons from human pluripotent stem cells using gene delivered transcription factor codes. Molecular therapy: the journal of the American Society of Gene Therapy 19, 1905-1912.
Hong, E. J., McCord, A. E., and Greenberg, M. E. (2008). A biological function for the neuronal activity-dependent component of Bdnf transcription in the development of cortical inhibition. Neuron 60, 610-624.
Hu, B. Y., and Zhang, S. C. (2009). Differentiation of spinal motor neurons from pluripotent human stem cells. Nature protocols 4, 1295-1304.
Imaizumi, Y., and Okano, H. (2014). Modeling human neurological disorders with induced pluripotent stem cells. Journal of neurochemistry 129, 388-399.
Ismadi, M. Z., Gupta, P., Fouras, A., Verma, P., Jadhav, S., Bellare, J., and Hourigan, K. (2014). Flow characterization of a spinner flask for induced pluripotent stem cell culture application. PloS one 9, e106493.
Kanning, K. C., Kaplan, A., and Henderson, C. E. (2010). Motor neuron diversity in development and disease. Annual review of neuroscience 33, 409-440.
Ke, M. T., Fujimoto, S., and Imai, T. (2013). SeeDB: a simple and morphology-preserving optical clearing agent for neuronal circuit reconstruction. Nature neuroscience 16, 1154-1161.
Kim, D. S., Ross, P. J., Zaslaysky, K., and Ellis, J. (2014). Optimizing neuronal differentiation from induced pluripotent stem cells to model ASD. Frontiers in cellular neuroscience 8, 109.
Lancaster, M. A., Renner, M., Martin, C. A., Wenzel, D., Bicknell, L. S., Hurles, M. E., Homfray, T., Penninger, J. M., Jackson, A. P., and Knoblich, J. A. (2013). Cerebral organoids model human brain development and microcephaly. Nature 501, 373-379.
Li, X. J., Zhang, X., Johnson, M. A., Wang, Z. B., Lavaute, T., and Zhang, S. C. (2009). Coordination of sonic hedgehog and Wnt signaling determines ventral and dorsal telencephalic neuron types from human embryonic stem cells. Development 136, 4055-4063.
Lin, Y., Bloodgood, B. L., Hauser, J. L., Lapan, A. D., Koon, A. C., Kim, T. K., Hu, L. S., Malik, A. N., and Greenberg, M. E. (2008). Activity-dependent regulation of inhibitory synapse development by Npas4. Nature 455, 1198-1204.
Liu, Y., and Zhang, S. C. (2010). Human stem cells as a model of motoneuron development and diseases. Annals of the New York Academy of Sciences 1198, 192-200.
Livesey, M. R., Bilican, B., Qiu, J., Rzechorzek, N. M., Haghi, G., Burr, K., Hardingham, G. E., Chandran, S., and Wyllie, D. J. (2014). Maturation of AMPAR composition and the GABAAR reversal potential in hPSC-derived cortical neurons. The Journal of neuroscience: the official journal of the Society for Neuroscience 34, 4070-4075.
Maroof, A. M., Keros, S., Tyson, J. A., Ying, S. W., Ganat, Y. M., Merkle, F. T., Liu, B., Goulburn, A., Stanley, E. G., Elefanty, A. G., et al. (2013). Directed differentiation and functional maturation of cortical interneurons from human embryonic stem cells. Cell stem cell 12, 559-572.
Otsuji, T. G., Bin, J., Yoshimura, A., Tomura, M., Tateyama, D., Minami, I., Yoshikawa, Y., Aiba, K., Heuser, J. E., Nishino, T., et al. (2014). A 3D sphere culture system containing functional polymers for large-scale human pluripotent stem cell production. Stem cell reports 2, 734-745.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Pasca, A. M., Sloan, S. A., Clarke, L. E., Tian, Y., Makinson, C. D., Huber, N., Kim, C. H., Park, J. Y., O'Rourke, N. A., Nguyen, K. D., et al. (2015). Functional cortical neurons and astrocytes from human pluripotent stem cells in 3D culture. Nature methods.
Qu, Q., Li, D., Louis, K. R., Li, X., Yang, H., Sun, Q., Crandall, S. R., Tsang, S., Zhou, J., Cox, C. L., et al. (2014). High-efficiency motor neuron differentiation from human pluripotent stem cells and the function of Islet-1. Nature communications 5, 3449.
Sasai, Y. (2013). Next-generation regenerative medicine: organogenesis from stem cells in 3D culture. Cell stem cell 12, 520-530.
Shi, Y., Kirwan, P., Smith, J., Robinson, H. P., and Livesey, F. J. (2012). Human cerebral cortex development from pluripotent stem cells to functional excitatory synapses. Nature neuroscience 15, 477-486, 5471.
Takazawa, T., Croft, G. F., Amoroso, M. W., Studer, L., Wichterle, H., and Macdermott, A. B. (2012). Maturation of spinal motor neurons derived from human embryonic stem cells. PloS one 7, e40154.
Vierbuchen, T., Ostermeier, A., Pang, Z. P., Kokubu, Y., Sudhof, T. C., and Wernig, M. (2010). Direct conversion of fibroblasts to functional neurons by defined factors. Nature 463, 1035-1041.
Wichterle, H., Lieberam, I., Porter, J. A., and Jessell, T. M. (2002). Directed differentiation of embryonic stem cells into motor neurons. Cell 110, 385-397.
Yang, Y. M., Gupta, S. K., Kim, K. J., Powers, B. E., Cerqueira, A., Wainger, B. J., Ngo, H. D., Rosowski, K. A., Schein, P. A., Ackeifi, C. A., et al. (2013). A small molecule screen in stem-cell-derived motor neurons identifies a kinase inhibitor as a candidate therapeutic for ALS. Cell stem cell 12, 713-726.
Zeng, H., Guo, M., Martins-Taylor, K., Wang, X., Zhang, Z., Park, J. W., Zhan, S., Kronenberg, M. S., Lichtler, A., Liu, H. X., et al. (2010). Specification of region-specific neurons including forebrain glutamatergic neurons from human induced pluripotent stem cells. PloS one 5, e11853.

1. A method of generating functional neurons from stem cells, the method comprising contacting a population of stem cells in spin culture with at least one differentiation medium to induce the differentiation of at least one sphere in the spin culture into a functional neuron differentiation sphere.

2. The method of claim 1, wherein the functional neuron differentiation sphere is a motor neuron differentiation sphere or a cortical neuron differentiation sphere.

3. (canceled)

4. The method of claim 1, wherein the differentiation medium is a neural induction medium.

5. The method of claim 1, wherein the differentiation medium includes a supplemental agent.

6. The method of claim 5, wherein the supplemental agent is selected from the group consisting of retinoic acid, brain-derived neurotrophic factor, smoothened agonist, DAPT and combinations thereof.

7. A method of generating functional cortical neurons from stem cells, the method comprising contacting a population of stem cells in spin culture with at least one differentiation medium to induce the differentiation of at least one sphere in the spin culture into a functional cortical neuron differentiation sphere.

8. The method of claim 7, wherein the at least one differentiation medium includes dual SMAD inhibition.

9. The method of claim 7, wherein a first differentiation medium includes a KSR medium or wherein a first differentiation medium includes a supplemental agent.

10. (canceled)

11. The method of claim 9, wherein a first differentiation medium includes a supplemental agent that is a Wnt signaling inhibitor.

12. The method of claim 7, wherein a second differentiation medium includes a neural induction medium.

13. (canceled)

14. The method of claim 9, wherein the population of stem cells in spin culture is further contacted with a neural induction medium.

15. The method of claim 7, further comprising maintaining the functional cortical neuron spheres in a neurobasal medium.

16. A method of generating functional motor neurons from stem cells, the method comprising contacting a population of stem cells in spin culture with at least one differentiation medium to induce the differentiation of at least one sphere in the spin culture into a functional motor neuron differentiation sphere.

17. The method of claim 16, wherein the at least one differentiation medium includes dual SMAD inhibition.

18. The method of claim 16, wherein a first differentiation medium includes a KSR medium, or wherein a first differentiation medium includes a supplemental agent.

19. (canceled)

20. The method of claim 18, wherein a first differentiation medium includes a supplemental agent selected from the group consisting of retinoic acid, brain-derived neurotrophic factor, smoothened agonist, DAPT and combinations thereof.

21. The method of claim 16, wherein a second differentiation medium includes a neural induction medium.

22. (canceled)

23. The method of claim 18, wherein the population of stem cells in spin culture are further contacted with a neural induction medium including at least one supplemental agent selected from the group consisting of retinoic acid, brain-derived neurotrophic factor, smoothened agonist, DAPT and combinations thereof.

24. The method of claim 16, further comprising maintaining the functional motor neuron spheres in a neurobasal medium including at least one supplemental agent selected from the group consisting of brain-derived neurotrophic factor, glial cell-derived neurotrophic factor, ciliary neurotrophic factor and combinations thereof.

25. A method for screening for neurodegenerative diseases comprising:
generating a neuron composition comprising a functional neuron from patient-derived induced pluripotent stem cells; and
screening for dysregulation of spontaneous activity or defects of stimulus-induced activity.
